(19) United States
(12) Patent Application Publication
(10) Pub. No.: US 2023/0357813 A1
(43) Pub. Date: Nov. 9, 2023

(54) HYPERSIALYLATED IMMUNOGLOBULIN

(71) Applicant: Momenta Pharmaceuticals, Inc., Titusville, NJ (US)

(72) Inventors: John Schaeck, Somerville, MA (US); Nathaniel Washburn, Littleton, MA (US)

(21) Appl. No.: 18/022,061

(22) PCT Filed: Aug. 20, 2021
(86) PCT No.: PCT/IB2021/057659

§ 371 Date: Feb. 17, 2023

Related U.S. Application Data
(60) Provisional application No. 63/068,796, filed on Aug. 21, 2020.

(51) Int. Cl. C12P 21/00; C07K 16/00; C12N 9/10

(52) U.S. Cl. CPC C12P 21/005; C07K 16/00; C12Y 204/99001; C12N 9/1081; C07K 2317/41

(57) ABSTRACT

Described herein are methods of preparing a hypersialylated human immunoglobulin G (hsIgG) preparations.

### CLAIM OF PRIORITY

This application claims the benefit of U.S. Provisional Application Ser. No. 63/068,796, filed on Aug. 21, 2020. The entire contents of the foregoing are incorporated herein by reference.

### TECHNICAL FIELD

Described herein are methods of preparing a hypersialylated human immunoglobulin G (hsIgG) preparations.

### BACKGROUND

The identification of the important role of Fc domain sialylation has presented an opportunity to develop potent immunoglobulin therapies. One commercially available source of immunoglobulins is intravenous immunoglobuin (IVIg), which is prepared from the pooled plasma of human donors (e.g., pooled plasma from at least 1,000 donors) and used to treat a variety of inflammatory disorders. Commercially available IVIg preparations generally exhibit low levels of sialylation on the Fc domain of the antibodies present. Specifically, they exhibit low levels of di-sialylation of the branched glycans on the Fc region. Further, IVIg preparations have distinct limitations, such as variable efficacy, high costs, and limited supply.

### SUMMARY

Described herein, inter alia, are methods for preparing immunoglobulin G (IgG) having a high level of Fc sialylation, in some embodiments, from fractionated blood plasma. The methods described herein can provide hypersialylated IgG (hsIgG) in which greater than 50% of the branched glycans on the Fc domain are sialylated on both branches (i.e., on the alpha 1,3 branch and the alpha 1,6 branch).

HsIgG contains a diverse mixture of IgG antibodies, primarily IgG1 antibodies. The diversity of the antibodies is high. The immunoglobulins used to prepare hsIgG using the methods described herein can be obtained, for example from pooled human plasma (e.g., pooled plasma from at least 1,000-30,000 donors). The immunoglobulins can be obtained from a variety of sources, including: pooled human plasma or serum, Cohn II/III fraction, Cohn I/II/III fraction, plasma from which cryoprecipitate has been removed, and ethanol precipitate of pooled human serum or plasma. The methods are useful for sialylating pooled IgG that are in compositions that include other proteins, e.g., other proteins that are present in human serum or plasma. For example, the methods described herein are useful for sialylating pooled IgG that is in a composition that is at least 5% (10%, 20%, 30%, 40% or more) wt/wt proteins that are not IgG. Thus the methods are useful for sialylating pooled IgG in a composition wherein less than 95% (90%, 80%, 70%, or 60%) wt/wt of the protein is IgG. The other proteins present in the composition can be non-IgG proteins that are present in human plasma or serum. The methods permit the efficient use of starting materials that are less pure than, for example, commercially available IVIg.

HsIgG has a far higher level of sialic acid on the branched glycans on the Fc region than does IVIg. This results in a composition that differs from IVIg in both structure and activity. HsIgG can be prepared as described in WO2014/179601 or Washburn et al. ((Proceedings of the National Academy of Sciences, USA 112: E1297-E1306 (2015)), both of which are hereby incorporated by reference in their entirety for any and all purposes. In many cases, hsIgG has a high level of disialylation of the branched glycans present on the Fab region.

Gamma globulins are known to be concentrated in the Plasma Fraction II-III of Cohn et al., J Clin. Invest. 23, 417-32 (1944); 1 Amer. Chem. Soc. 68, 459-75 (1946); called “Cohn II/III” or “Cohn II,III” interchangeably throughout this application. In some embodiments, fractionation of blood plasma yields gamma globulin concentrate. In some embodiments, described herein are methods of preparing a hsIgG preparation from a Cohn II/III fraction. In some embodiments, described herein are methods of preparing a hsIgG preparation from Cohn II/III. In some embodiments, described herein are methods of preparing a hsIgG preparation from Cohn II/III soluble portion (Cohn IV/V). HsIgG can also be prepared using plasma from which cryoprecipitate has been removed (sometimes called cryosupernatant, cryopoor plasma, cryoprecipitate depleted plasma or cryoprecipitate reduced plasma).

Described herein are methods of preparing a hypersialylated human immunoglobulin G (hsIgG) preparation comprising: (a) providing a composition comprising pooled human immunoglobulin G (IgG) wherein at least 5% or at least 10% wt/wt of protein in the composition is not IgG;(b) adding β1,4-Galactosyltransferase I (β4GalT) and uridine 5′-diphosphogalactose (UDP-Gal), together or sequentially, to the composition to create a reaction mixture, wherein the reaction mixture is in a buffer; (c) incubating the reaction mixture; (d) adding ST6 beta-galactoside alpha-2,6-sialyltransferase 1 (ST6Gal1) and cytidine-5′-monophospho-N-acetylneuraminic acid (CMP-NANA), together or sequentially, to the reaction mixture; and (e) incubating the reaction mixture, thereby creating the hsIgG preparation.

In some embodiments, step (c) is carried out for at least 8, 12, 18, 24, 30, 40, or 18-22 hrs and step (e) is carried out for at least 8, 12, 18, 24, 30, 40, or 30-32 hrs.

In some embodiments, step (d) comprises adding ST6Gal1 and CMP-NANA to the reaction mixture of step (a).

Also described herein are methods of preparing hypersialylated (hsIgG) comprising: (a) providing a composition comprising pooled human IgG wherein at least 5% or at least 10% wt/wt of protein in the composition is not IgG in a buffer;(b) incubating the composition in a reaction mixture comprising β1,4-Galactosyltransferase I (β4GalT), UDP-Gal, ST6Gal1 and CMP-NANA, in a buffer, thereby creating the hsIgG preparation.

Also described herein are methods of preparing hypersialylated (hsIgG) comprising: (a) providing a composition comprising pooled human IgG wherein at least 5% or at least 10% wt/wt of protein in the composition is not IgG; (b) combining the composition with β4GalT and ST6Gal to create a reaction mixture, wherein the reaction mixture is in a buffer and contains UDP-Gal and CMP-NANA; and incubating the reaction mixture, thereby creating the hsIgG preparation.

In some embodiments, the buffer is selected from the group consisting of Bis(2-hydroxyethyl)amino0tris(hydroxymethyl)methane (BIS-TRIS), 3-(N-morpholino)propanesulfonic acid (MOPS), 2-(N-morpholino)ethanesulfonic acid (MES), 1,4-Piperazinediethanesulfonic acid (PIPES), N,N-Bis(2-hydroxyethyl)-2-aminoethanesulfonic acid (BES), 3-morpholino-2-hydroxypropanesulfonic acid (MOPSO), Triethanolamine (TEA), Piperazine-N—N′-bis(2-hydroxypropanesulfonic acid (POPSO), 4-(2-Hydroxyethyl)-1-piperazinepropanesulfonic acid (EPPS), and combinations thereof.

In some embodiments, providing a mixture of IgG antibodies includes (a) providing pooled plasma from at least 1000 human subjects; and (b) isolating a mixture of IgG antibodies from the pooled plasma.

In some embodiments, β4GalT and ST6Gal and added at the same time or sequentially in either order. In some embodiments, additional CMP-NANA is added to the reaction at a time after the first addition of CMP-NANA. In some embodiments, additional CMP-NANA is added to the reaction at a time after the second addition of CMP-NANA. In some embodiments, the first and second addition of CMP-NANA are separated by 2, 3, 4, 5, 6, 7, 8, 9, 10, 11, 12, 13, or 14 hours. In some embodiments, the second and third addition of CMP-NANA are separated by 2, 3, 4, 5, 6, 7, 8, 9, 10, 11, 12, 13, 14, 15, 16, 17, or 18 hours.

In some embodiments, β1,4-Galactosyltransferase I (β4GalT) and UDP-Gal are added to the reaction at the same time. In some embodiments, β1,4-Galactosyltransferase I (β4GalT) and UDP-Gal are not added to the reaction at the same time. In some embodiments, ST6Gal1 and CMP-NANA are added to the reaction at the same time. In some embodiments, ST6Gal1 and CMP-NANA are not added to the reaction at the same time.

In some embodiments, the method further comprises subjecting the hsIgG preparation to one or more purification steps.

In some embodiments, incubating the reaction mixture comprising β4GalT is carried out under conditions and for a time that permits galactosylation of branched glycans present on the human IgG. In some embodiments, incubating the reaction mixture comprising ST6Gal1 is carried out under conditions and for a time that permits sialylation of galactosylated branched glycans present on the human IgG.

In some embodiments, at least 60%, 70%, 80% or 90% wt/wt of the protein in the composition comprising pooled human immunoglobulin G (IgG) is IgG. In some embodiments, at least 10%, 15%, 20% or 30% wt/wt of the protein in the composition comprising pooled human immunoglobulin G (IgG) is not IgG.

In some embodiments, the composition comprising pooled human IgG is a composition selected from the group consisting of: pooled human plasma, pooled human serum, cryoprecipitate depleted plasma, pooled human serum or plasma that has been ethanol precipitated, human Cohn II/III plasma fraction, or human Cohn plasma I/II/III fraction. In some embodiments, the composition comprising pooled human IgG is human Cohn II/III fraction or human Cohn I/II/III fraction.

In some embodiments, the step of providing the human Cohn II/III plasma fraction or the human Cohn I/II/III plasma fraction comprises cold ethanol precipitation of proteins from human serum pooled from at least 100 donors, at least 500 donors or at least 1000 donors. In some embodiments, the step of providing the human Cohn II/III plasma fraction further comprises one or more of: precipitation, chromatography, filtration, delipidation, pathogen inactivation, and combinations thereof.

In some embodiments, the step of providing a composition comprising pooled human IgG comprises exchanging a composition comprising pooled human IgG into a buffer or solubilizing a composition comprising pooled human IgG in a buffer. In some embodiments, the buffer is BIS-TRIS.

In some embodiments, the β4GalT1 comprises an amino acid sequence that is at least 85, 90, 95, 96, 97, 98, 99, or 100% identical to SEQ ID NO: 12 or SEQ ID NO: 13 and the ST6Gal1 comprises an amino acid sequence that is at least 85, 90, 95, 96, 97, 98, 99, or 100% identical to SEQ ID NO: 14, SEQ ID NO: 15, SEQ ID NO: 18, SEQ ID NO: 19, SEQ ID NO: 20, SEQ ID NO: 21, the portion of SEQ ID NO: 19 from amino acid 23 to 320, the portion of SEQ ID NO: 19 from amino acid 13 to 320, the portion of SEQ ID NO: 19 from amino acid 11 to 320, the portion of SEQ ID NO: 19 from amino acid 6 to 320, the portion of SEQ ID NO: 19 from amino acid 5 to 320, or the portion of SEQ ID NO: 19 from amino acid 4 to 320.

In some embodiments, the salt concentration in the reaction mixtures is below 150 mM, below 125 mM, below 100 mM, below 50 mM, below 25 mM, below 10 mM or below 5 mM.

In some embodiments, incubating the reaction mixture comprising β4GalT is carried out for at least 8, 12, 18, 24, 30, 40, or 18-22 hrs and incubating the reaction mixture comprising ST6Gal1 is carried out for at least 8, 12, 18, 24, 30, 40, or 30-32 hrs.

In some embodiments, the step of providing the Cohn II/III plasma fraction or the Cohn I/II/III plasma fraction in a buffer comprises: solubilizing the Cohn II/III or the Cohn I/II/III material in a buffer, removing undissolved material and subjection the resulting solution to buffer exchange into a buffer. In some embodiments, the buffer is BIS-TRIS. In some embodiments, the method further comprises adding one or more of: a delipidation agent, a high purity diatomite filter media, or a fumed silica to the solution prior to buffer exchange. In some embodiments, one of more of calcium silicate hydrate, silicon dioxide and fumed silica are added to the solution prior to buffer exchange. In some embodiments, the method further comprises depth filtering the solution.

In some embodiments, the reactions take place at pH 5.5-8.5. In some embodiments, the reactions take place in BIS-TRIS at 10-500 mM pH 5.5-8.5.

In some embodiments, the reaction mixtures comprise MnCl₂ at 1-20 mM.

In some embodiments, the initial concentration of UDP-Gal in the reaction mixture comprising UDP-Gal is 20-500 μmol UDP-Gal/g IgG antibody. In some embodiments, the initial concentration of CMP-NANA in the reaction mixture comprising CMP-NANA is 100-3000 μmol CMP-NANA/g IgG antibody.

In some embodiments, the incubation takes place at 20-50° C. In some embodiments, the incubation takes place at 30-45° C. or 35-39° C.

In some embodiments, the IgG antibodies comprise IgG antibodies isolated from at least 1000 donors.

In some embodiments, at least 50%, 55%, 60%, 65% or 70% w/w of the IgG antibodies are IgG1 antibodies.

In some embodiments, the hsIgG preparation is further treated to removed ST6Gal1 and β4GalT.

In some embodiments, about 50%, 55%, 60%, 65%, 70%, 75%, 80%, or 85% of the branched glycans in the hsIgG preparation have a sialic acid on both the α1,3 branch and the α1,6 branch. In some embodiments, about 50%, 55%, 60%, 65%, 70%, 75%, 80%, or 85% of the branched Fc glycans in the hsIgG preparation have a sialic acid on both the α1,3 branch and the α1,6 branch. In some embodiments, at least 50%, 55%, 60%, 65%, 70%, 75%, 80%, or 85% of the branched glycans on the Fab domain have a sialic acid on both the α 1,3 arm and the α 1,6 arm that is connected through a NeuAc-α 2,6-Gal terminal linkage. In some embodiments, least 50%, 55%, 60%, 65%, 70%, 75%, 80%, or 85% of the branched glycans on the Fc domain have a sialic acid on both the α 1,3 arm and the α 1,6 arm that is connected through a NeuAc-α 2,6-Gal terminal linkage.

In some embodiments, incubating the reaction mixture comprising β4GalT is carried out from 12-30 hours. In some embodiments, incubating the reaction mixture comprising ST6Gal1 is carried out from 20-40 hours.

In some embodiments, the method further comprises one or more additional purification steps, wherein non-IgG proteins are reduced or removed. In some embodiments, the protein that is not IgG is protein present in human plasma or serum.

In some embodiments, β4GalT is present in the reaction mixture comprising β4GalT at greater than 5, 10, 20, 30, 50, or 100 mU/mg IgG. In some embodiments, ST6Gal is present in the reaction mixture comprising ST6Gal1 at greater than 5, 10, 20, 50, 100 or 200 mU/mg IgG.

In some embodiments, greater than 50%, 55%, 60%, 65%, 70%, 75%, 80%, or 85% of the branched glycans on the IgG antibodies in the hsIgG preparation have a sialic acid on both the α1,3 branch and the α1,6 branch.

In some embodiments, incubating the reaction mixture comprising β4GalT proceeds for a period of time sufficient to produce galactosylated IgG antibodies. In some embodiments, incubating the reaction mixture comprising ST6Gal1 proceeds for a period of time sufficient to produce disialylated IgG antibodies.

Also described herein is a method of preparing hypersialylated (hsIgG), the method comprising: (a) providing a composition comprising pooled human plasma, pooled human serum, cryoprecipitate depleted pooled human plasma, an ethanol precipitate of pooled human serum or plasma, a Cohn II/III fraction of pooled human plasma or serum, a Cohn IV/V fraction of pooled human serum or plasma or a Cohn I/II/III fraction of pooled human serum or plasma; (b) incubating the composition in a reaction mixture comprising β1,4-Galactosyltransferase I (β4GalT) and UDP-Gal to produce galactosylated IgG antibodies; (c) incubating the galactosylated IgG antibodies in a reaction mixture comprising ST6Gal1 and CMP-NANA, wherein the galactosylation reaction mixture and the sialylation reaction mixture comprise Bis (2-hydroxyethyl) aminotris (hydroxymethyl)methane (BIS-TRIS) buffer, thereby creating the hsIgG preparation.

Also described is a method of preparing hypersialylated (hsIgG), the method comprising (a) providing a composition comprising pooled human plasma, pooled human serum, cryoprecipitate depleted pooled human plasma, an ethanol precipitate of pooled human serum or plasma a Cohn II/III fraction of pooled human plasma or serum, a Cohn IV/V fraction of pooled human serum or plasma or a Cohn I/II/III fraction of pooled human serum or plasma; (b) incubating the composition in a reaction mixture comprising β1,4-Galactosyltransferase I (β4GalT), UDP-Gal, ST6Gal1 and CMP-NANA, in Bis (2-hydroxyethyl) aminotris (hydroxymethyl)methane (BIS-TRIS) buffer, for at least 24 hours, thereby creating the hsIgG preparation.

Described herein is a method of preparing hypersialylated (hsIgG), the method comprising: (a) providing a composition comprising pooled human IgG wherein at least 5% (10%, 20% 30%, 40% or 50%) wt/wt of the protein in the composition is not IgG; (b) incubating the composition in a reaction mixture comprising β1,4-Galactosyltransferase I (β4GalT) and UDP-Gal to produce galactosylated IgG antibodies; (c) incubating the galactosylated IgG antibodies in a reaction mixture comprising ST6Gal1 and CMP-NANA, wherein the galactosylation reaction mixture and the sialylation reaction mixture comprise Bis (2-hydroxyethyl) aminotris (hydroxymethyl)methane (BIS-TRIS) buffer, thereby creating the hsIgG preparation.

Also described is a method of preparing hypersialylated (hsIgG), the method comprising (a) providing a composition comprising pooled human IgG wherein at least 5% (10%, 20%, 30%, 40% or 50%) wt/wt of the protein in the composition is not IgG; (b) incubating the composition in a reaction mixture comprising β1,4-Galactosyltransferase I (β4GalT), UDP-Gal, ST6Gal1 and CMP-NANA, in Bis (2-hydroxyethyl) aminotris (hydroxymethyl)methane (BIS-TRIS) buffer, for at least 24 hours, thereby creating the hsIgG preparation.

In some embodiments of any of the methods described herein, the Cohn II/III fraction is a lyophilized solid obtained by ethanol precipitation of pooled plasma which is then resuspended in BIS-TRIS. In some embodiments of any of the methods described herein, the Cohn II/III fraction is a lyophilized solid obtained by ethanol precipitation of pooled plasma which is then buffer exchanged into BIS-TRIS prior to incubating the mixture of IgG antibodies in a reaction mixture.

In various embodiments: the β4GalT1 is at least 80, 85, 90, 95, 96, 97, 98, 99, or 100% identical to SEQ ID NO: 12 or SEQ ID NO: 13; the ST6Gal1 comprises an amino acid sequence that is at least 80, 85, 90, 95, 96, 97, 98, 99, or 100% identical to SEQ ID NO: 14 or SEQ ID NO: 19; step (b) is carried out for at least 8, 12, 18, 24, 30, or 40 hrs; step (c) is carried out for at least 8, 12, 18, 24, 30, or 40 hrs; step (c) comprises adding ST6Gal1 and CMP-NANA to the reaction mixture of step (a); the reactions take place in BIS-TRIS at 10-500 mM pH 5.5-8.5; the reaction mixtures comprise MnCl₂ at 1-20 mM; the reaction takes place in 25-75 mM pH 6.5-7.3 BIS-TRIS, 2.5-7.5 mM MnCl₂; the UDP-Gal is present at 95 μmol UDP-Gal/g protein; the UDP-Gal is present at 40-200 μmol UDP-Gal/g protein; the CMP-NANA is present at 700 μmol CMP-NANA/g protein; the CMP-NANA is present at 200-2200 μmol CMP-NANA/g protein; the β4GalT is present at 23 U B4GalT/g protein; the β4GalT is present at 10-95 U B4GalT/g protein; the ST6Gal1 is present at 94 U ST6/g protein; the ST6Gal1 is present at 20-300 U ST6/g protein; the incubation takes place at 20-50° C.; the incubation takes place at 30-45° C.; the IgG antibodies comprise IgG antibodies isolated from at least 1000 donors; at least 70%, 80%, 90% w/w of the IgG antibodies are IgG1 antibodies; at least 90% of the donor subjects have been exposed to a virus; about 60%, 65%, 70%, 75%, 80%, or 85% of the branched glycans in the hsIgG preparation have a sialic acid on both the α1,3 branch and the α1,6 branch; about 60%, 65%, 70%, 75%, 80%, or 85% of the branched Fc glycans in the hsIgG preparation have a sialic acid on both the α1,3 branch and the α1,6 branch; at least 60%, 65%, 70%, 75%, 80%, or 85% of the branched glycans on the Fab domain have a sialic acid on both the α 1,3 arm and the α 1,6 arm that is connected through a NeuAc-α 2,6-Gal terminal linkage; at least 60%, 65%, 70%, 75%, 80%, or 85% of the branched glycans on the Fc domain have a sialic acid on both the a 1,3 arm and the α 1,6 arm that is connected through a NeuAc-α 2,6-Gal terminal linkage; the incubation in step (b) is 12-30 hours; and the incubation in step (c) is 20-40 hours.

In the case of compositions in which greater than 97% of the protein is IgG: UDP-Gal is present at 15-60 umol/gm protein (e.g., 38 umol UDP-Gal/g protein), CMP-NANA is present at 110-600 umol/g protein (e.g., 220 umol/g protein); and 4-20 U B4GalT/g protein (e.g., 7.5 U/g protein), 7.5-45 U ST6/g protein (e.g., 15 U/g protein range).

In some embodiments, a composition comprises sialylated IgG comprising sialylated IgG with at least 50% of the glycans are sialylated on both the α1,3 branch and the α1,6 branch. In some embodiments, at least 50%, 55%, 60%, 65%, 70%, 75%, 80%, or 85% of the Fc glycans are sialylated on both the α1,3 branch and the α1,6 branch. In some embodiments, at least 50%, 55%, 60%, 65%, 70%, 75%, 80%, or 85% of the Fc glycans are sialylated on both the α1,3 branch and the α1,6 branch and at least 50%, 55%, 60%, 65%, 70%, 75%, 80%, or 85% of the Fab glycans are sialylated on both the α1,3 branch and the α1,6 branch.

In some embodiments, the immunoglobulins are derived from fractionated plasma, e.g., human plasma pooled from at least 100, 500 or 1,000 donors. In certain embodiments, more than 50%, 55%, 60%, 65%, 70%, 75%, 80%, 85%, 90%, or 95% the immunoglobulins are IgG polypeptides (e.g., IgG1, IgG2, IgG3 or IgG4 or mixtures thereof), although amounts of other immunoglobulin subclasses can be present.

In some embodiments, any of the methods described herein can further comprise one or more additional purification steps, wherein non-IgG proteins are reduced or removed

In some embodiments, the invention relates to a method for treating a disorder, the method comprising administering a composition comprising hsIgG to a subject at a dose that alleviates at least one symptom associated with the disorder. In some embodiments, a composition comprising hsIgG is administered at a dose of about 4, 5, 6, 10, 15, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 975, or 1000 mg/kg. In some embodiments, a composition comprising sialylated hsIgG is administered daily, weekly, semiweekly, biweekly, monthly, semimonthly, bimonthly, every 3 days, every 4 days, every 5 days, every 6 days, every 7 days, once every 14 days, once every 21 days, once every 28 days, once daily for two consecutive days in a 28-day cycle, or with the same administration frequency as the FDA approved IVIG dose. In some embodiments, a composition comprising hsIgG is administered intravenously, subcutaneously, or intramuscularly. In some embodiments, a composition is administered in a single dose. In some embodiments, a composition is administered in multiple doses.

In some embodiments, the disorder is an inflammatory disorder. In some embodiments, the disorder is associated with the presence of autoantibodies.

In some embodiments, the disorder is a neurological disorder. In some embodiments, the neurological disorder is selected from the group consisting of: dermatomyositis, Guillain-Barre syndrome, chronic inflammatory demyelinating polyneuropathy (CIDP), multifocal motor neuropathy (MMN), myasthenia gravis and stiff person syndrome.

In some embodiments, the disorder is selected from the group consisting of: sculitis, systemic lupus erythematosis (SLE), mucous membrane pemphigoid and uveitis and in dermatology it is used most commonly to treat Kawasaki syndrome, dermatomyositis, toxic epidermal necrolysis and the blistering diseases.

In some embodiments, the disorder is FDA-approved for treatment with IVIG. In some embodiments, the dose is less than, about equal to the FDA approved IVIG dose for the disorder. In some embodiments, the FDA approved dose of IVIG is 200 mg/kg, 400 mg/kg, 500 mg/kg, 600 mg/kg, 1000 mg/kg, or 2000 mg/kg. In some embodiments, a composition comprising hsIgG is administered at a dose of about 4, 5, 6, 10, 15, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 975, or 1000 mg/kg. In some embodiments, a composition comprising hsIgG is administered at a fraction of the FDA approved IVIG dose for the disorder, e.g., % 1/2, 1/3, 1/4, 1/5, 1/6, 1/7, 1/8, 1/9, or 1/10th the FDA approved dose for the disorder. In some embodiments, a composition comprising hsIgG is administered daily, weekly, semiweekly, biweekly, monthly, semimonthly, bimonthly, every 3 days, every 4 days, every 5 days, every 6 days, every 7 days, once every 14 days, once every 21 days, once every 28 days, once daily for two consecutive days in a 28-day cycle, or with the same administration frequency as the FDA approved IVIG dose. In some embodiments, a composition comprising hsIgG is administered intravenously, subcutaneously, or intramuscularly. In some embodiments, a composition is administered in a single dose. In some embodiments, a composition is administered in multiple doses.

In some embodiments, the disorder is selected from the group consisting of: Myocarditis, Acute motor axonal neuropathy, Adiposis dolorosa, Anti-Glomerular Basement Membrane nephritis; Goodpasture syndrome, Antiphospholipid syndrome (APS, APLS), Antisynthetase syndrome; Myositis, ILD, ataxic neuropathy (acute & chronic), Autoimmune enteropathy (AIE), Autoimmune neutropenia, Autoimmune retinopathy, Autoimmune thyroiditis, Autoimmune urticaria, Dermatitis herpetiformis, Epidermolysis bullosa acquisita, Essential mixed cryoglobulinemia, Granulomatosis with polyangiitis (GPA), Mixed connective tissue disease (MCTD), Neuromyotonia, Optic neuritis, Paraneoplastic cerebellar degeneration, Anti-N-Methyl-D-Aspartate (Anti-NMDA) Receptor Encephalitis, Autoimmune hemolytic anemia, Autoimmune thrombocytopenic purpura, immune thrombocytopenia, Chronic inflammatory demyelinating polyneuropathy, Dermatomyositis, Gestational pemphigoid, Graves' disease, Guillain-Barré syndrome, IgG4-related disease, Lambert-Eaton myasthenic syndrome, Lupus nephritis, Myositis, Multifocal motor neuropathy, Myasthenia gravis, Neuromyelitis optica, Pemphigus vulgaris, Polymyositis, Systemic Lupus Erythematosus (SLE), and combinations thereof.

In some embodiments, the disorder is selected from the group consisting of Acute disseminated encephalomyelitis (ADEM), Autoimmune Angioedema (Acquired angioedema type II), Autoimmune hepatitis (Type I & Type II), Autoimmune hypophysitis; Lymphocytic hypophysitis, Autoimmune inner ear disease (AIED), Evans syndrome, Graves ophthalmopathy, Hashimoto's encephalopathy, IgA vasculitis (IgAV), Latent autoimmune hepatitis, Linear IgA disease (LAD), Lupus vasculitis, Membranous glomerulonephritis, Microscopic polyangiitis (MPA), Mooren's ulcer, Morphea, Opsoclonus myoclonus syndrome, Ord's thyroiditis, Palindromic rheumatism, Paraneoplastic opsoclonus—myoclonus-ataxia with neuroblastoma, Pediatric Autoimmune Neuropsychiatric Disorder Associated with Streptococcus (PANDAS), Postpericardiotomy syndrome, Primary biliary cirrhosis (PBC), Rasmussen Encephalitis, Rheumatoid vasculitis, Schnitzler syndrome, Sydenham chorea, Undifferentiated connective tissue disease (UCTD), Miller Fisher Syndrome, and combinations thereof.

In some embodiments, a composition comprises hsIgG wherein at least 50% of the branched glycans on the Fab domain are sialylated on both the α 1,3 arm and the α 1,6 arm by way of NeuAc-α 2,6-Gal terminal linkages; and at least 50% of the branched glycan on the Fc domain are sialylated on both the a 1,3 arm and the α 1,6 arm by way of NeuAc-α 2,6-Gal terminal linkages.

In some embodiments, an invention relates to a method of treating CIDP in a subject having CIDP comprising administering hsIgG at or less than an effective dose for IVIG. In some embodiments, the effective dose for IVIG is 200-2000 mg/kg. In some embodiments, the hsIgG is administered at a dose of 10, 15, 20, 25, 30, 35, 40, 45, 50, 55, 60, 65, 70, 75, 80, 90, or 100% of the effective dose for IVIG. In some embodiments, the hsIgG is administered at a dose of about 2, 3, 4, 5, 6, 7, 8, 9, 10, 15, 20, 25, 30, 35, 40, 45, 50, 55, 60, 65, 70, 75, 80, 90, 100, 110, 120, 130, 140, 150, 160, 170, 180, 190, or 200 mg/kg.

In some embodiments, an invention relates to a method of treating immune thrombocytopenia in a subject having immune thrombocytopenia comprising administering hsIgG at or less than an effective dose effective dose for IVIG. In some embodiments, the effective dose for IVIG is 1000-2000 mg/kg mg/kg. In some embodiments, the hsIgG is administered at a dose of 10, 15, 20, 25, 30, 35, 40, 45, 50, 55, 60, 65, 70, 75, 80, 90, or 100% of the effective dose for IVIG. In some embodiments, the hsIgG is administered at a dose of about 10, 20, 30, 40, 50, 60, 70, 80, 90, 100, 110, 120, 130, 140, 150, 160, 170, 180, 190, or 200 mg/kg.

In some embodiments, an invention relates to a method of treating wAIHA in a subject having wAIHA comprising administering hsIgG at or less than an effective dose for IVIG. In some embodiments, the effective dose for IVIG is 1000 mg/kg. In some embodiments, the hsIgG is administered at a dose of 10, 15, 20, 25, 30, 35, 40, 45, 50, 55, 60, 65, 70, 75, 80, 90, or 100% of the effective dose for IVIG. In some embodiments, the hsIgG is administered at a dose of about 10, 20, 30, 40, 50, 60, 70, 80, 90, or 100 mg/kg.

In some embodiments, an invention relates to a method of treating Guillain-Barre Syndrome in a subject having Guillain-Barre Syndrome comprising administering hsIgG at or less than an effective dose for IVIG. In some embodiments, the effective dose for IVIG is 1000-2000 mg/kg. In some embodiments, the hsIgG is administered at a dose of 10, 15, 20, 25, 30, 35, 40, 45, 50, 55, 60, 65, 70, 75, 80, 90, or 100% of the effective dose for IVIG. In some embodiments, the hsIgG is administered at a dose of about 10, 20, 30, 40, 50, 60, 70, 80, 90, 100, 110, 120, 130, 140, 150, 160, 170, 180, 190, or 200 mg/kg.

In some embodiments, an invention relates to a method of treating PID (primary humoral immunodeficiency disease) in a subject having PID comprising administering hsIgG at or less than an effective dose for IVIG. In some embodiments, the effective dose for IVIG is 200-800 mg/kg. In some embodiments, the hsIgG is administered at a dose of 10, 15, 20, 25, 30, 35, 40, 45, 50, 55, 60, 65, 70, 75, 80, 90, or 100% of the effective dose for IVIG. In some embodiments, the hsIgG is administered at a dose of about 2, 3, 4, 5, 6, 7, 8, 9, 10, 15, 20, 25, 30, 35, 40, 45, 50, 55, 60, 65, 70, 75, or 80 mg/kg.

In some embodiments, an invention relates to a method of treating Kawasaki disease in a subject having Kawasaki disease comprising administering hsIgG at or less than an effective dose for IVIG. In some embodiments, the effective dose for IVIG is 1000-2000 mg/kg. In some embodiments, the hsIgG is administered at a dose of 10, 15, 20, 25, 30, 35, 40, 45, 50, 55, 60, 65, 70, 75, 80, 90, or 100% of the effective dose for IVIG. In some embodiments, the hsIgG is administered at a dose of about 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 975, or 1000 mg/kg.

In some embodiments, administering a composition comprising hsIgG has similar efficacy to administering a composition comprising the effective dose of IVIG.

In some embodiments, the composition comprises polypeptides that are derived from plasma, e.g., human plasma. In certain embodiments, the polypeptides are overwhelmingly IgG polypeptides (e.g., IgG1, IgG2, IgG3 or IgG4 or mixtures thereof), although trace amounts of other contain trace amount of other immunoglobulin subclasses can be present.

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

As used herein, the term “constant region” refers to a polypeptide that corresponds to, or is derived from, one or more constant region immunoglobulin domains of an antibody. A constant region can include any or all of the following immunoglobulin domains: a C_{H}1 domain, a hinge region, a C_{H}2 domain, a C_{H}3 domain (derived from an IgA, IgD, IgG, IgE, or IgM), and a C_{H}4 domain (derived from an IgE or IgM).

As used herein, the term “Fc region” refers to a dimer of two “Fc polypeptides,” each “Fc polypeptide” including the constant region of an antibody but excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or the entire flexible hinge N-terminal to these domains. For IgG, “Fc polypeptide” comprises immunoglobulin domains Cgamma2 (Cγ2) and Cgamma3 (Cγ3) and the lower part of the hinge between Cgamma1 (Cγ1) and Cγ2. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, VA). For IgA, Fc polypeptide comprises immunoglobulin domains Calpha2 (Cα2) and Calpha3 (Cα3) and the lower part of the hinge between Calpha1 (Cα1) and Cα2. An Fc region can be synthetic, recombinant, or generated from natural sources such as IVIg.

As used herein, “glycan” is a sugar, which can be monomers or polymers of sugar residues, such as at least three sugars, and can be linear or branched. A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a polypeptide, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties. In some cases, the term “glycoprotein” herein refers to immunoglobulin that is covalently linked to one or more sugar moieties.

As used herein, “IVIg” is a preparation of pooled, polyvalent IgG, including all four IgG subgroups, extracted from plasma of at least 1,000 human donors. IVIg is approved as a plasma protein replacement therapy for immune deficient patients. The level of IVIg Fc glycan sialylation varies among IVIg preparations, but is generally less than 20%. The level of disialylation is generally far lower. As used herein, the term “derived from IVIg” refers to polypeptides which result from manipulation of IVIg. For example, polypeptides purified from IVIg (e.g., enriched for sialylated IgGs) or modified IVIg (e.g., IVIg IgGs enzymatically sialylated).

As used herein, an “N-glycosylation site of an Fc polypeptide” refers to an amino acid residue within an Fc polypeptide to which a glycan is N-linked. In some embodiments, an Fc region contains a dimer of Fc polypeptides, and the Fc region comprises two N-glycosylation sites, one on each Fc polypeptide.

As used herein “percent (%) of branched glycans” refers to the number of moles of glycan X relative to total moles of glycans present, wherein X represents the glycan of interest.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

“Pharmaceutical preparations” and “pharmaceutical products” can be included in kits containing the preparation or product and instructions for use.

“Pharmaceutical preparations” and “pharmaceutical products” generally refer to compositions in which the final predetermined level of sialylation has been achieved, and which are free of process impurities. To that end, “pharmaceutical preparations” and “pharmaceutical products” are substantially free of ST6Ga1 sialyltransferase and/or sialic acid donor (e.g., cytidine 5′-monophospho-N-acetyl neuraminic acid) and/or the byproducts thereof (e.g., cytidine 5′-monophosphate).

“Pharmaceutical preparations” and “pharmaceutical products” are generally substantially free of other components of a cell in which the glycoproteins were produced (e.g., the endoplasmic reticulum or cytoplasmic proteins and RNA), if recombinant.

By “purified” (or “isolated”) refers to a polynucleotide or a polypeptide that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acids. An isolated polynucleotide or polypeptide can be at least 60% free, or at least 75% free, or at least 90% free, or at least 95% free from other components present in natural environment of the indicated polynucleotide or polypeptide.

As used herein, the term “sialylated” refers to a glycan having a terminal sialic acid. The term “mono-sialylated” refers to branched glycans having one terminal sialic acid, e.g., on an α1,3 branch or an α1,6 branch. The term “di-sialylated” refers to a branched glycan having a terminal sialic acid on two arms, e.g., both an α1,3 arm and an α1,6 arm.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the protein distribution in Cohn II/III and Privigen®.

FIG. 2 shows the non-IgG protein distribution in Cohn II/III and Privigen®. Based on normalized peptide spectral matches, Cohn II/III was about 65% IgG and 35% non-IgG, which is consistent with reported values. Privigen® was greater than 99% IgG.

FIG. 3 shows a short, branched core oligosaccharide comprising two N-acetylglucosamine and three mannose residues. One of the branches is referred to in the art as the “a 1,3 arm,” and the second branch is referred to as the “a 1,6 arm,”. Squares: N-acetylglucosamine; dark gray circles: mannose; light gray circles: galactose; diamonds: N-acetylneuraminic acid; triangles: fucose.

FIG. 4 shows common Fc glycans present in IVIg. Squares: N-acetylglucosamine; dark gray circles: mannose; light gray circles: galactose; diamonds: N-acetylneuraminic acid; triangles: fucose.

FIG. 5 shows how immunoglobulins, e.g., IgG antibodies, can be sialylated by carrying out a galactosylation step followed by a sialylation step. Squares: N-acetylglucosamine; dark gray circles: mannose; light gray circles: galactose; diamonds: N-acetylneuraminic acid; triangles: fucose.

FIG. 6 shows the reaction product of a representative example of the IgG-Fc glycan profile for a reaction starting with IVIg. The glycans were quantified by LCMS analysis of glycopeptides from a trypsin digestion of the IgGs. Due to human population genetic heterogeneity the IgG3 glycopeptides are identical to either IgG2 or IgG4 and hence are quantified together. The left panel is a schematic representation of enzymatic sialylation reaction to transform IgG to hsIgG; the right panel is the IgG Fc glycan profile for the starting IVIg and hsIgG. Bars, from left to right, correspond to IgG1, IgG2/3, and IgG3/4, respectively.

FIG. 7 shows structures of fully galactosylated and sialylated glycans.

FIG. 8 shows fraction of IgG1 branched glycans that are disialylated in the sialylation reactions (Table 11) on Aldrich Cohn II, III material which was directly dissolved in BIS-TRIS. The glycans were quantified by LCMS analysis of glycopeptides from a trypsin digestion of the IgGs. Disialylated is defined as the sum A2F, A2F+bisect, A2.

FIG. 9 shows fraction of IgG1 branched glycans that are disialylated in the sialylation reactions (Table 12) on Aldrich Cohn II, III material which was buffer exchanged into BIS-TRIS. The glycans were quantified by LCMS analysis of glycopeptides from a trypsin digestion of the IgGs. Disialylated is defined as the sum A2F, A2F+bisect, A2.

FIG. 10 shows fraction of IgG2/3 branched glycans that are disialylated in the sialylation reactions (Table 11) on Aldrich Cohn II, III material which was directly dissolved in BIS-TRIS. The glycans were quantified by LCMS analysis of glycopeptides from a trypsin digestion of the IgGs. Disialylated is defined as the sum A2F, A2F+bisect, A2.

FIG. 11 shows fraction of IgG3/4 branched glycans that are disialylated in the sialylation reactions (Table 11) on Aldrich Cohn II, III material which was directly dissolved in BIS-TRIS. The glycans were quantified by LCMS analysis of glycopeptides from a trypsin digestion of the IgGs. Disialylated is defined as the sum A2F, A2F+bisect, A2.

FIG. 12 shows fraction of IgG2/3 branched glycans that are disialylated in the sialylation reactions (Table 12) on Aldrich Cohn II, III material which was buffer exchanged into BIS-TRIS. The glycans were quantified by LCMS analysis of glycopeptides from a trypsin digestion of the IgGs. Disialylated is defined as the sum A2F, A2F+bisect, A2.

FIG. 13 shows fraction of IgG3/4 branched glycans that are disialylated in the sialylation reactions (Table 12) on Aldrich Cohn II, III material which was buffer exchanged into BIS-TRIS. The glycans were quantified by LCMS analysis of glycopeptides from a trypsin digestion of the IgGs. Disialylated is defined as the sum A2F, A2F+bisect, A2.

FIG. 14 shows an SDS-PAGE gel comparing the protein purity profile of Aldrich Cohn II/III material to a soluble fraction of Cohn II/III paste.

### DETAILED DESCRIPTION

The present disclosure encompasses, in part, methods for preparing immunoglobulins (e.g., human IgG) having an Fc region having particular levels of branched glycans that are sialylated on both of the arms of the branched glycan (e.g., with a NeuAc-α 2,6-Gal terminal linkage). The levels can be measured on an individual Fc region (e.g., the number of branched glycans that are sialylated on an α1,3 arm, an α1,6 arm, or both, of the branched glycans in the Fc region), or on the overall composition of a preparation of polypeptides (e.g., the number or percentage of branched glycans that are sialylated on an α1,3 arm, an α1,6 arm, or both, of the branched glycans in the Fc region in a preparation of polypeptides). In some embodiments, the present disclosure concerns methods of treatment using hsIgG.

Immunoglobulins are glycosylated at conserved positions in the constant regions of their heavy chain. For example, IgG antibodies have a single N-linked glycosylation site at Asn297 of the C_{H}2 domain. For human IgG, which is the type of antibody present in IVIG and hsIgG, the core oligosaccharide normally consists of GlcNAc2Man3GlcNAc and a core fucose, with differing numbers of outer residues. Variation among individual IgG's can occur via attachment of galactose and/or galactose-sialic acid at one or both terminal GlcNAc or via attachment of a third GlcNAc arm (bisecting GlcNAc). Various commercial preparations of IVIG are general less than 10% sialylated or even less than 5% sialylated on the Asn297 of the C_{H}2 domain.

Naturally derived polypeptides that can be used to prepare hypersialylated IgG include, for example, IgG in human serum (e.g., human serum pooled from more than 1,000 donors), IgG derived from human serum, intravenous immunoglobulin (IVIg) and polypeptides derived from IVIg (e.g., polypeptides purified from IVIg (e.g., enriched for sialylated IgGs)) or modified IVIg (e.g., IVIg IgGs enzymatically sialylated).

The level of sialylation can be measured on an individual Fc region (e.g., the number of branched glycans that are sialylated on an α1,3 arm, an α1,6 arm, or both, of the branched glycans in the Fc region), or on the overall composition of a preparation of glycoproteins (e.g., the number or percentage of branched glycans that are sialylated on an α1,3 arm, an α1,6 arm, or both, of the branched glycans in the Fc region in a preparation of glycoproteins).

Hypersialylated IgG is a composition comprising immunoglobulin in which at least 40% of the glycans in the Fc regions are disialylated, i.e., have a sialic acid on the α1,3 arm (e.g., with a NeuAc-α2,6-Gal terminal linkage) and on the α1,6 arm (e.g., with a NeuAc-α2,6-Gal terminal linkage).

In some embodiments, a sialylation level is an absolute level or range of (e.g., number of moles of or percent of) one or more glycans (e.g., branched glycans having a sialic acid on an α1,3 arm, and/or branched glycans having a sialic acid on an α1,6 arm, and/or branched glycans having a sialic acid on an α1,3 arm and on an α1,6 arm) in a glycoprotein preparation. In some embodiments, a predetermined or target level is a level or range of one or more glycans (e.g., branched glycans having a sialic acid on an α1,3 arm, and/or branched glycans having a sialic acid on an α1,6 arm, and/or branched glycans having a sialic acid on an α1,3 arm and on an α1,6 arm) in a glycoprotein preparation relative to total level of glycans in the glycoprotein preparation. In some embodiments, a predetermined or target level is a level or range of one or more glycans (e.g., branched glycans having a sialic acid on an α1,3 arm, and/or branched glycans having a sialic acid on an α1,6 arm, and/or branched glycans having a sialic acid on an α1,3 arm and on an α1,6 arm) in a glycoprotein preparation relative to total level of sialylated glycans in the glycoprotein preparation. In some embodiments, a predetermined or target level is expressed as a percent.

ST6 beta-galactoside alpha-2,6-sialyltransferase 1 (ST6 or ST6Gal1) (e.g., human ST6) is used in the methods described herein. Useful ST6 includes polypeptides whose amino acid sequence includes at least one characteristic sequence of and/or shows at least 100%, 99%, 98%, 97%, 96%, 95%, 94%, 93%, 92%, 91%, 90%, 89%, 88%, 87%, 86%, 85%, 84%, 83%, 82%, 81%, 80%, or 70% identity with a protein involved in transfer of a sialic acid to a terminal galactose of a glycan through an α2,6 linkage (e.g., human ST6Gal1 or ST6), e.g., polypeptides that are at least 100%, 99%, 98%, 97%, 96%, 95%, 94%, 93%, 92%, 91%, 90%, 89%, 88%, 87%, 86%, 85%, 84%, 83%, 82%, 81%, 80%, or 70% identity to any of SEQ ID NOs:1 and 4-9. A wide variety of ST6 sialyltransferase sequences are known in the art, such as those described herein; in some embodiments, an ST6 sialyltransferase shares at least one characteristic sequence of and/or shows the specified degree of overall sequence identity with one of the ST6 sialyltransferases set forth herein (each of which may be considered a “reference” ST6 sialyltransferase). In some embodiments, an ST6 sialyltransferase as described herein shares at least one biological activity with a reference ST6 sialyltransferase as set forth herein. In some such embodiment, the shared biological activity relates to transfer of a sialic acid to a glycan. In some embodiments, an ST6 sialyltransferase is a human sialyltransferase.

Beta-1,4-galactosyltransferase 1 (B4GalT), e.g., human B4GalT, is used in the methods described herein. Useful B4GalT can be a polypeptide whose amino acid sequence includes at least one characteristic sequence of and/or shows at least 100%, 99%, 98%, 97%, 96%, 95%, 94%, 93%, 92%, 91%, 90%, 89%, 88%, 87%, 86%, 85%, 84%, 83%, 82%, 81%, 80%, or 70% identity with a protein involved in transfer of a transfers galactose from uridine 5′-diphosphosegalactose (UDP-Gal) to GlcNAc as a β-1,4 linkage. A wide variety of Beta-1,4-galactosyltransferase sequences are known in the art, such as those described herein; in some embodiments, an Beta-1,4-galactosyltransferase shares at least one characteristic sequence of and/or shows the specified degree of overall sequence identity with one of the Beta-1,4-galactosyltransferases set forth herein (each of which may be considered a “reference” Beta-1,4-galactosyltransferase). In some embodiments, an Beta-1,4-galactosyltransferase as described herein shares at least one biological activity with a reference Beta-1,4-galactosyltransferase as set forth herein. In some such embodiment, the shared biological activity relates to transfer of a galactose to a glycan. In some embodiments, an Beta-1,4-galactosyltransferase is a human galactosyltransferase.

Cohn II/III and/or Cohn I/II/III can be used as a source of IgG to prepare hsIgG. Various methods for preparing Cohn II/III and/or Cohn I/II/III are described in Cohn et al. (J. Amer. Chem. Soc. 68, 459-75 (1946)). For example, Cohn Fraction I is precipitated from pooled plasma and removed by adding ethanol at low temperatures (e.g., at 0.027 mole fraction to plasma at −3° C.). The pH of the supernatant (Cohn Fraction II/III/IV/V) is reduced by adding a buffer and salt (e.g., the pH of the supernatant is reduced to about 6.8 by addition of sodium acetate-acetic acid buffer with a molar ratio of salt to acid of 1.77) and precipitation at −5° C. with 0.091 mole fraction in ethanol yielding Cohn II/III.

Cohn II/III can be purchased from Sigma-Aldrich (St. Louis, MO) and various suppliers of plasma products.

In some embodiments, a first fractionation/precipitation can be performed to precipitate Cohn Fraction I leaving all other components soluble. A subsequent precipitation can yield Cohn II/III. In some embodiments, an initial fractionation can yield Cohn I/II/III.

### Enzymes

### Galactosylating Enzymes

Beta-1,4-galactosyltransferase (B4GalT), e.g., human B4GalT, e.g., human B4Galt1, as well as orthologs, mutants, and variants thereof, including enzymatically active portions of beta-1,4-galactosyltransferase (B4GalT), e.g., human B4GalT, e.g., human B4Galt1, as well as orthologs, mutants, and variants thereof, along with fusion proteins and polypeptides comprising the same are suitable for use in the methods described herein. B4Galt1 is one of seven beta-1,4-galactosyltransferase (beta4GalT) genes that each encode type II membrane-bound glycoproteins that appear to have exclusive specificity for the donor substrate UDP-galactose; all transfer galactose in a beta 1,4 linkage to similar acceptor sugars: GlcNAc, Glc, and Xyl. B4Galt1 adds galactose to N-acetylglucosamine residues that are either monosaccharides or the nonreducing ends of glycoprotein carbohydrate chains. B4GalT1 is also called GGTB2. Four alternative transcripts encoding four isoforms of B4GALT1 (NCBI Gene ID 2683) are described in Table 1.

TABLE 1
Human B4GALT1 isoforms

| | Length | | | Length | |
|---|---|---|---|---|---|
| Transcript | (nt) | Protein | SEQ ID NO: | (aa) | Isoform |
| NM_001497.4 | 4176 | NP_001488.2 | SEQ ID NO: 5 | 398 | 1 |
| NM_001378495.1 | 3999 | NP_001365424.1 | SEQ ID NO: 6 | 385 | 2 |
| NM_001378496.1 | 4053 | NP_001365425.1 | SEQ ID NO: 7 | 357 | 3 |
| NM_001378497.1 | 1520 | NP_001365426.1 | SEQ ID NO: 8 | 225 | 4 |

TABLE 2
Topology of B4GALT1 isoform 1 (SEQ ID NO: 5)

| | | | | | SEQ |
|---|---|---|---|---|---|
| Feature | AAs | Description | Length | Sequence | ID NO: |
| Topologica | 1-24 | Cytoplasmic | 9 | MRLREPLLSGSAAMPGASLQR | SEQ |
| 1 domain | | | | ACR | ID NO: |
| | | | | | 9 |
| Trans- | 25-44 | Helical; | 17 | LLVAVCALHLGVTLVYYLAG | SEQ |
| membrane | | Signal- | | | ID NO: |
| | | anchor for | | | 10 |
| | | type II | | | |
| | | membrane | | | |
| | | protein | | | |
| Topological | 45-398 | Lumenal | 380 | RDLSRLPQLVGVSTPLQGGSN | SEQ |
| domain | | | | SAAAIGQSSGELRTGGARPPP | ID NO: |
| | | | | PLGASSQPRPGGDSSPVVDSG | 11 |
| | | | | PGPASNLTSVPVPHTTALSLP | |
| | | | | ACPEESPLLVGPMLIEFNMPV | |
| | | | | DLELVAKQNPNVKMGGRYAPR | |
| | | | | DCVSPHKVAIIIPFRNRQEHL | |
| | | | | KYWLYYLHPVLQRQQLDYGIY | |
| | | | | VINQAGDTIFNRAKLLNVGFQ | |
| | | | | EALKDYDYTCFVFSDVDLIPM | |
| | | | | NDHNAYRCFSQPRHISVAMDK | |
| | | | | FGFSLPYVQYFGGVSALSKQQ | |
| | | | | FLTINGFPNNYWGWGGEDDDI | |
| | | | | FNRLVFRGMSISRPNAVVGRC | |
| | | | | RMIRHSRDKKNEPNPQRFDRI | |
| | | | | AHTKETMLSDGLNSLTYQVLD | |
| | | | | VQRYPLYTQITVDIGTPS | |

TABLE 3
Binding sites of B4GALT1 isoform 1 (SEQ ID NO: 5)

| Position(s) | Description | Reference(s) |
|---|---|---|
| 250 | Metal binding; | |
| | Manganese | |
| 310 | Binding site; | “Structural snapshots of beta-1,4- |
| | UDP-alpha-D- | galactosyltransferase-I along the kinetic pathway.” |
| | galactose | Ramakrishnan B., Ramasamy V., Qasba P. K. |
| | | J. Mol. Biol. 357: 1619-1633(2006) |
| 343 | Metal binding; | |
| | Manganese; via | |
| | tele nitrogen | |
| 355 | Binding site; N- | “Oligosaccharide preferences of beta1,4- |
| | acetyl-D- | galactosyltransferase-I: crystal structures of |
| | glucosamine | Met340His mutant of human beta1,4- |
| | | galactosyltransferase-I with a pentasaccharide and |
| | | trisaccharides of the N-glycan moiety.” |
| | | Ramasamy V., Ramakrishnan B., Boeggeman E., |
| | | Ratner D. M., Seeberger P. H., Qasba P. K. |
| | | J. Mol. Biol. 353: 53-67(2005) |
| | | “Deoxygenated disaccharide analogs as specific |
| | | inhibitors of beta1-4-galactosyltransferase 1 and |
| | | selectin-mediated tumor metastasis.” |
| | | Brown J. R., Yang F., Sinha A., Ramakrishnan B., |
| | | Tor Y., Qasba P. K., Esko J. D. |
| | | J. Biol. Chem. 284: 4952-4959(2009) |

TABLE 4
Post Translational Amino Acid Modifications of B4GALT1 isoform 1 (SEQ ID NO: 5)

| Feature key | Position(s) | Description | Reference(s) |
|---|---|---|---|
| Glycosylation | 113 | N-linked | |
| | | (GlcNAc . . .) | |
| | | asparagine | |
| Disulfide | 130 ↔ 172 | | “Oligosaccharide preferences of beta1,4- |
| bond | | | galactosyltransferase-I: crystal structures of |
| Disulfide | 243 ↔ 262 | | Met340His mutant of human beta1,4- |
| bond | | | galactosyltransferase-I with a |
| | | | pentasaccharide and trisaccharides of the |
| | | | N-glycan moiety.” |
| | | | Ramasamy V., Ramakrishnan B., |
| | | | Boeggeman E., Ratner D. M., Seeberger |
| | | | P. H., Qasba P. K. |
| | | | J. Mol. Biol. 353: 53-67(2005) |
| | | | “Structural snapshots of beta-1,4- |
| | | | galactosyltransferase-I along the kinetic |
| | | | pathway.” |
| | | | Ramakrishnan B., Ramasamy V., |
| | | | Qasba P. K. |
| | | | J. Mol. Biol. 357: 1619-1633(2006) |

The soluble form of B4GalT1 derives from the membrane form by proteolytic processing. The cleavage site is at positions 77-78 of B4GALT1 isoform 1 (SEQ ID NO: 5).

In some embodiments, one or more of the amino acids of the B4GalT1 corresponding to amino acids 113, 130, 172, 243, 250, 262, 310, 343, or 355 of B4GALT1 isoform 1 (SEQ ID NO: 5) is conserved as compared to (SEQ ID NO: 5). In some embodiments, the enzyme is an enzymatically active portion of, e.g., B4GalT1. In some embodiments, the enzyme is an enzymatically active portion of B4GALT1 isoform 1 (SEQ ID NO: 5), or an ortholog, mutant, or variant of SEQ ID NO: 5. In some embodiments, the enzyme is an enzymatically active portion of B4GALT1 isoform 2 (SEQ ID NO: 6), or an ortholog, mutant, or variant of SEQ ID NO: 6. In some embodiments, the enzyme is an enzymatically active portion of B4GALT1 isoform 3 (SEQ ID NO: 7), or an ortholog, mutant, or variant of SEQ ID NO: 7. In some embodiments, the enzyme is an enzymatically active portion of B4GALT1 isoform 4 (SEQ ID NO: 8), or an ortholog, mutant, or variant of SEQ ID NO: 8.

In some embodiments, the enzymatically active portion of B4GalT1 does not comprise a cytoplasmic domain, e.g., SEQ ID NO: 9. In some embodiments, the enzymatically active portion of B4GalT1 does not comprise a transmembrane domain, e.g., SEQ ID NO: 10. In some embodiments, the enzymatically active portion of B4GalT1 does not comprise a cytoplasmic domain, e.g., SEQ ID NO: 9 or a transmembrane domain, e.g., SEQ ID NO: 10.

In some embodiments, the enzymatically active portion of B4GalT1 comprises all or a portion of a luminal domain, e.g., SEQ ID NO: 11, or an ortholog, mutants, or variants thereof.

In some embodiments, the enzymatically active portion of B4GalT1 comprises amino acids 109-398 of SEQ ID NO: 5, or an ortholog, mutants, or variants thereof. In some embodiments, the enzymatically active portion of B4GalT1 consists of SEQ ID NO: 5, or an ortholog, mutant, or variant of SEQ ID NO: 5.

A suitable functional portion of an B4GalT1 can comprise or consist of an amino acid sequence that is at least 80% (85%, 90%, 95%, 98% or 100%) identical to SEQ ID NO: 12.

Also suitable for use in the methods described herein is an amino acid sequence that comprises or consists of an amino acid sequence that is at least 80% (85%, 90%, 95%, 98% or 100%) identical to SEQ ID NO: 13.

### Sialylating Enzymes

ST6, e.g., ST6Gal1, e.g., human ST6Gal1, as well as orthologs, mutants, and variants thereof, including enzymatically active portions of ST6Gal1, e.g., human ST6Gal1, as well as orthologs, mutants, and variants thereof, along with fusion proteins and polypeptides comprising the same, are suitable for use in the methods described herein. Alpha-2,6-sialyltransferase 1 (ST6) is a Type II Golgi membrane-bound glycoprotein that transfers sialic acid from cytidine 5′-monophospho-N-acetylneuraminic acid (CMP-NANA) to Gal as an α-2,6 linkage. ST6Gal1 is also called as ST6N or SIAT1. Four alternative transcripts encoding two isoforms of ST6GAL1 (NCBI Gene ID 6480) are described in Table 5.

TABLE 5
Human ST6GAL1 isoforms

| | Length | | | Length | |
|---|---|---|---|---|---|
| Transcript | (nt) | Protein | SEQ ID NO: | (aa) | Isoform |
| NM_173216.2 | 4604 | NP_775323.1 | SEQ ID NO: 14 | 406 | a |
| NM_173217.2 | 3947 | NP_775324.1 | SEQ ID NO: 15 | 175 | b |
| NM_003032.3 | 4303 | NP_003023.1 | SEQ ID NO: 14 | 406 | a |
| NM_001353916.2 | 4177 | NP_001340845.1 | SEQ ID NO: 14 | 406 | a |

TABLE 6
Topology of ST6Gall isoform a (SEQ ID NO: 14)

| | | | | | SEQ |
|---|---|---|---|---|---|
| | | | | | ID |
| Feature | AAS | Description | Length | Sequence | NO: |
| Topological | 1-9 | Cytoplasmic | 9 | MIHTNLKKK | SEQ |
| domain | | | | | ID |
| | | | | | NO: |
| | | | | | 16 |
| Trans- | 10-26 | Helical; | 17 | FSCCVLVFLLFAVICVW | SEQ |
| membrane | | Signal- | | | ID |
| | | anchor for | | | NO: |
| | | type II | | | 17 |
| | | membrane | | | |
| | | protein | | | |
| Topological | 27-406 | Lumenal | 380 | KEKKKGSYYDSFKLQTKEFQVLK | SEQ |
| domain | | | | SLGKLAMGSDSQSVSSSSTQDPH | ID |
| | | | | RGRQTLGSLRGLAKAKPEASFQV | NO: |
| | | | | WNKDSSSKNLIPRLQKIWKNYLS | 18 |
| | | | | MNKYKVSYKGPGPGIKFSAEALR | |
| | | | | CHLRDHVNVSMVEVTDFPFNTSE | |
| | | | | WEGYLPKESIRTKAGPWGRCAVV | |
| | | | | SSAGSLKSSQLGREIDDHDAVLR | |
| | | | | FNGAPTANFQQDVGTKTTIRLMN | |
| | | | | SQLVTTEKRFLKDSLYNEGILIV | |
| | | | | WDPSVYHSDIPKWYQNPDYNFFN | |
| | | | | NYKTYRKLHPNQPFYILKPQMPW | |
| | | | | ELWDILQEISPEEIQPNPPSSGM | |
| | | | | LGIIIMMTLCDQVDIYEFLPSKR | |
| | | | | KTDVCYYYQKFFDSACTMGAYHP | |
| | | | | LLYEKNLVKHLNQGTDEDIYLLG | |
| | | | | KATLPGFRTIHC | |

TABLE 7
Binding sites of ST6Gal1 isoform a (SEQ ID NO: 14)

| Position(s) | Description | Reference(s) |
|---|---|---|
| 189 | Substrate; via | “The structure of human alpha-2,6-sialyltransferase |
| | amide nitrogen | reveals the binding mode of complex glycans.” |
| 212 | Substrate | Kuhn B., Benz J., Greif M., Engel A. M., Sobek H., |
| 233 | Substrate | Rudolph M. G. Acta Crystallogr. D 69: 1826- |
| 353 | Substrate; via | 1838(2013) |
| | carbonyl oxygen | |
| 354 | Substrate | |
| 365 | Substrate | |
| 369 | Substrate | |
| 370 | Substrate | “The structure of human alpha-2,6-sialyltransferase |
| 376 | Substrate | reveals the binding mode of complex glycans.” |
| | | Kuhn B., Benz J., Greif M., Engel A. M., Sobek H., |
| | | Rudolph M. G. Acta Crystallogr. D 69: 1826- |
| | | 1838(2013) |

TABLE 8
Post Translational Amino Acid Modifications
of ST6Gal1 isoform a (SEQ ID NO: 14)

| Feature key | Position(s) | Description | Reference(s) |
|---|---|---|---|
| Disulfide | 142 ↔ 406 | | “The structure of human alpha-2,6- |
| bond | | | sialyltransferase reveals the binding |
| | | | mode of complex glycans.” |
| | | | Kuhn B., Benz J., Greif M., Engel |
| | | | A. M., Sobek H., Rudolph M. G. |
| | | | Acta Crystallogr. D 69: 1826- |
| | | | 1838(2013) |
| Glycosylation | 149 | N-linked | “Glycoproteomics analysis of human |
| | | (GlcNAc . . .) | liver tissue by combination of multiple |
| | | asparagine | enzyme digestion and hydrazide |
| | | | chemistry.” |
| | | | Chen R., Jiang X., Sun D., Han G., |
| | | | Wang F., Ye M., Wang L., Zou H. |
| | | | J. Proteome Res. 8: 651-661(2009); and |
| | | | “The structure of human alpha-2,6- |
| | | | sialyltransferase reveals the binding |
| | | | mode of complex glycans.” |
| | | | Kuhn B., Benz J., Greif M., Engel |
| | | | A. M., Sobek H., Rudolph M. G. |
| | | | Acta Crystallogr. D 69: 1826- |
| | | | 1838(2013) |
| Glycosylation | 161 | N-linked | “Glycoproteomics analysis of human |
| | | (GlcNAc . . .) | liver tissue by combination of multiple |
| | | asparagine | enzyme digestion and hydrazide |
| | | | chemistry.” |
| | | | Chen R., Jiang X., Sun D., Han G., |
| | | | Wang F., Ye M., Wang L., Zou H. |
| | | | J. Proteome Res. 8: 651-661(2009) |
| Disulfide | 184 ↔ 335 | | “The structure of human alpha-2,6- |
| bond | | | sialyltransferase reveals the binding |
| | | | mode of complex glycans.” |
| | | | Kuhn B., Benz J., Greif M., Engel |
| | | | A. M., Sobek H., Rudolph M. G. |
| | | | Acta Crystallogr. D 69: 1826- |
| | | | 1838(2013) |
| Disulfide | 353 ↔ 364 | | “The structure of human alpha-2,6- |
| bond | | | sialyltransferase reveals the binding |
| | | | mode of complex glycans.” |
| | | | Kuhn B., Benz J., Greif M., Engel |
| | | | A. M., Sobek H., Rudolph M. G. |
| | | | Acta Crystallogr. D 69: 1826- |
| | | | 1838(2013) |
| Modified | 369 | Phosphotyrosine | “Quantitative phosphoproteomic |
| residue | | | analysis of T cell receptor signaling |
| | | | reveals system-wide modulation of |
| | | | protein-protein interactions.” |
| | | | Mayya V., Lundgren D. H., Hwang |
| | | | S. I., Rezaul K., Wu L., Eng J. K., |
| | | | Rodionov V., Han D. K. |
| | | | Sci. Signal. 2: RA46-RA46(2009) |

The soluble form of ST6Gal1 derives from the membrane form by proteolytic processing.

In some embodiments, one or more of the amino acids of the ST6Gal1 corresponding to amino acids 142, 149, 161, 184, 189, 212, 233, 335, 353, 354, 364, 365, 369, 370, 376, or 406 of ST6Gal1 isoform a (SEQ ID NO: 14) is conserved as compared to SEQ ID NO: 14.

Also provided herein is an enzymatically active portion of, e.g., ST6Gal1. In some embodiments, the enzyme is an enzymatically active portion of STG6Gal1 isoform a (SEQ ID NO: 14), or an ortholog, mutant, or variant of SEQ ID NO: 14. In some embodiments, the enzyme is an enzymatically active portion of STG6Gal1 isoform b (SEQ ID NO: 15), or an ortholog, mutant, or variant of SEQ ID NO: 15.

In some embodiments, the enzymatically active portion of ST6Gal1 does not comprise a cytoplasmic domain, e.g., SEQ ID NO: 16. In some embodiments, the enzymatically active portion of ST6Gal1 does not comprise a transmembrane domain, e.g., SEQ ID NO: 17. In some embodiments, the enzymatically active portion of ST6Gal1 does not comprise a cytoplasmic domain, e.g., SEQ ID NO: 16 or a transmembrane domain, e.g., SEQ ID NO: 17.

In some embodiments, the enzymatically active portion of ST6Gal1 comprises all or a portion of a luminal domain, e.g., SEQ ID NO: 18, or an ortholog, mutants, or variants thereof.

In some embodiments, the enzymatically active portion of ST6Gal1 comprises amino acids 87-406 of SEQ ID NO: 14 (SEQ ID NO: 19), or an ortholog, mutants, or variants thereof. In some embodiments, the enzymatically active portion of ST6Gal1 consists of SEQ ID NO: 19, or an ortholog, mutant, or variant of SEQ ID NO: 19.

A suitable functional portion of an ST6Gal1 can comprise or consist of an amino acid sequence that is at least 70%, (80%, 85%, 90%, 95%, 98% or 100%) identical to SEQ ID NO: 19.

In some embodiments, the ST6Gal1 comprises or consists of SEQ ID NO: 19, the portion of SEQ ID NO: 19 from amino acid 23 to 320, the portion of SEQ ID NO: 19 from amino acid 13 to 320, the portion of SEQ ID NO: 19 from amino acid 11 to 320, the portion of SEQ ID NO: 19 from amino acid 6 to 320, the portion of SEQ ID NO: 19 from amino acid 5 to 320, or the portion of SEQ ID NO: 19 from amino acid 4 to 320.

In some embodiments, the enzymatically active portion of ST6Gal1 comprises amino acids 109-406 of SEQ ID NO: 14 (SEQ ID NO: 20), or an ortholog, mutants, or variants thereof. In some embodiments, the enzymatically active portion of ST6Gal1 consists of SEQ ID NO: 20, or an ortholog, mutant, or variant of SEQ ID NO: 20.

A suitable functional portion of an ST6Gal1 can comprise or consist of an amino acid sequence that is at least 70%, (80%, 85%, 90%, 95%, 98% or 100%) identical to SEQ ID NO: 20.

Also suitable for use in the methods described herein is an amino acid sequence that comprises or consists of an amino acid sequence that is at least 70% (80%, 85%, 90%, 95%, 98% or 100%) identical to SEQ ID NO: 21.

### Variants

In some embodiments, the enzyme(s) described herein are at least 80%, e.g., at least 85%, 90%, 95%, 98%, or 100% identical to the amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent, e.g., β-galactoside α-2,6-sialyltransferase activity or β-1,4-galactosyltransferase activity.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M.O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

In some embodiments, an enzyme that has a sequence that is not 100% identical to a sequence described herein differs by amino acid substitutions or deletions. In the case of substitution, the difference can be conservative or nonconservative substitution of one or more amino acid residues. Conservative substitutions are those that substitute a given amino acid in a polypeptide by another amino acid of similar characteristics. Typical conservative substitutions are the following replacements: replacement of an aliphatic amino acid, such as alanine, valine, leucine, and isoleucine, with another aliphatic amino acid; replacement of a serine with a threonine or vice versa; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

In some embodiments, a B4GalT or a ST6 sialyltransferase polypeptide includes a substituent group on one or more amino acid residues. Still other useful polypeptides are associated with (e.g., fused, linked, or coupled to) another moiety (e.g., a peptide or molecule). For example, a B4GalT or an ST6 sialyltransferase polypeptides can be fused, linked, or coupled to an amino acid sequence (e.g., a leader sequence, a secretory sequence, a proprotein sequence, a second polypeptide, or a sequence that facilitates purification, enrichment, or stabilization of the polypeptide).

### Glycans

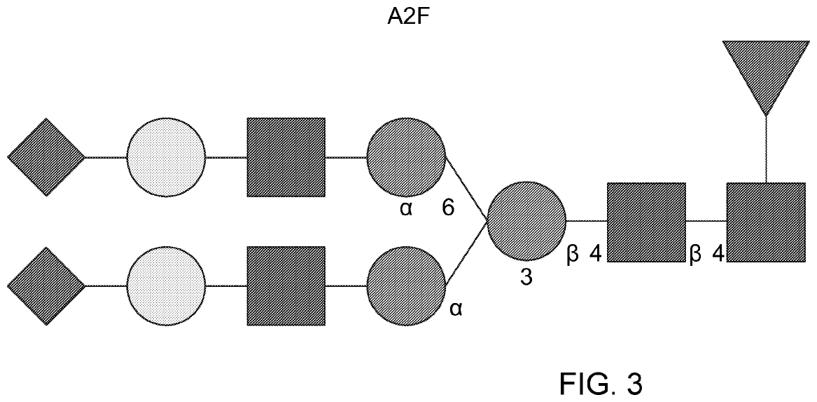

In vivo, N-linked oligosaccharide chains are added to a protein, for example an immunoglobulin, in the lumen of the endoplasmic reticulum. Specifically, an initial oligosaccharide (typically 14-sugar) is added to the amino group on the side chain of an asparagine residue contained within the target consensus sequence of Asn-X-Ser/Thr, where X may be any amino acid except proline. The structure of this initial oligosaccharide is common to most eukaryotes, and contains three glucose, nine mannose, and two N-acetylglucosamine residues. This initial oligosaccharide chain can be trimmed by specific glycosidase enzymes in the endoplasmic reticulum, resulting in a short, branched core oligosaccharide composed of two N-acetylglucosamine and three mannose residues. One of the branches is referred to in the art as the “α1,3 arm,” and the second branch is referred to as the “α1,6 arm,” as shown in FIG. 3.

N-glycans can be subdivided into three distinct groups called “high mannose type,” “hybrid type,” and “complex type,” with a common pentasaccharide core (Man (α1,6)-(Man(α1,3))-Man(β1,4)-GlcNAc(β1,4)-GlcNAc(β1,N)-Asn) occurring in all three groups.

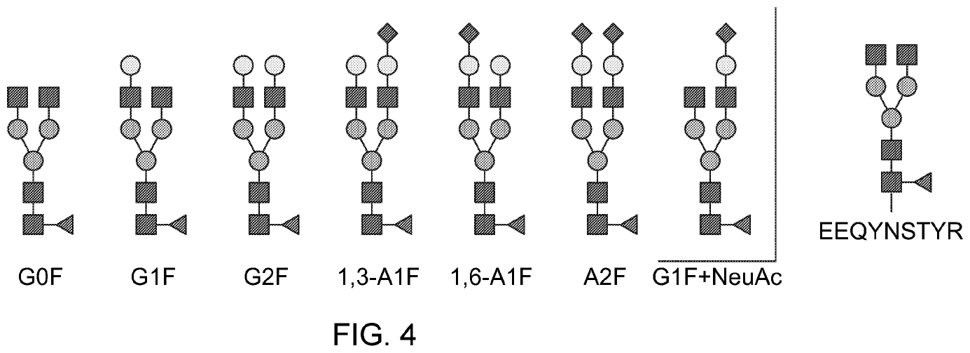

The more common Fc glycans present in IVIg include those shown in FIG. 4.

After initial processing in the endoplasmic reticulum, the polypeptide is transported to the Golgi where further processing may take place. If the glycan is transferred to the Golgi before it is completely trimmed to the core pentasaccharide structure, it results in a “high-mannose glycan.”

Additionally or alternatively, one or more monosaccharides units of N-acetylglucosamine may be added to the core mannose subunits to form a “complex glycan.” Galactose may be added to the N-acetylglucosamine subunits, and sialic acid subunits may be added to the galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

Sialic acids are a family of 9-carbon monosaccharides with heterocyclic ring structures. They bear a negative charge via a carboxylic acid group attached to the ring as well as other chemical decorations including N-acetyl and N-glycolyl groups. The two main types of sialyl residues found in polypeptides produced in mammalian expression systems are N-acetyl-neuraminic acid (NeuAc) and N-glycolylneuraminic acid (NeuGc). These usually occur as terminal structures attached to galactose (Gal) residues at the non-reducing termini of both N- and O-linked glycans. The glycosidic linkage configurations for these sialyl groups can be either α2,3 or α2,6.

Fc regions are glycosylated at conserved, N-linked glycosylation sites. For example, each heavy chain of an IgG antibody has a single N-linked glycosylation site at Asn297 of the C_{H}2 domain (see Jefferis, Nature Reviews 8:226-234 (2009)). IgA antibodies have N-linked glycosylation sites within the C_{H}2 and C_{H}3 domains, IgE antibodies have N-linked glycosylation sites within the C_{H}3 domain, and IgM antibodies have N-linked glycosylation sites within the C_{H}1, C_{H}2, C_{H}3, and C_{H}4 domains (see Arnold et al., J. Biol. Chem. 280:29080-29087 (2005); Mattu et al., J. Biol. Chem. 273:2260-2272 (1998); Nettleton et al., Int. Arch. Allergy Immunol. 107:328-329 (1995)).

Each antibody isotype has a distinct variety of N-linked carbohydrate structures in the constant regions. For example, IgG has a single N-linked biantennary carbohydrate at Asn297 of the C_{H}2 domain in each Fc polypeptide of the Fc region, which also contains the binding sites for C1q and FcγR (see Jefferis et al., Immunol. Rev. 163:59-76 (1998); and Wright et al., Trends Biotech 15:26-32 (1997)). For human IgG, the core oligosaccharide normally consists of GlcNAc2Man3GlcNAc, with differing numbers of outer residues. Variation among individual IgG can occur via attachment of galactose and/or galactose-sialic acid at one or both terminal GlcNAc or via attachment of a third GlcNAc arm (bisecting GlcNAc), and/or attachment of fucose.

### Antibodies

Antibodies useful in the methods described herein include, for example, immunoglobulins, e.g., IgG antibodies. The antibodies, e.g., IgG antibodies, can be pooled. For example, IgG antibodies can be in pooled plasma. In some embodiments, the IgG antibodies comprise IgG antibodies isolated from at least 1000 donors. In some embodiments, at least 50%, 55%, 60%, 65% or 70% w/w of the IgG antibodies are IgG1 antibodies. In some embodiments, at least 90% of the donor subject has been exposed to a virus.

In some cases, the pooled IgG antibodies are produced using fractionation, e.g., cold ethanol fractionation (e.g., the Cohn-Oncley procedure (see Cohn et al., “Preparation and Properties of Serum and Plasma Proteins: IV. A System for the Separation into Fractions of the Protein and Lipoprotein Components of Biological Tissues and Fluids,” J Am Chem Soc 68:459-75 (1946))). See, e.g., Ofosu et al., “Plasma-Derived Biological Medicines Used to Promote Haemostasis,” Thromb Haemost 99:851-62 (2008); see also Cai et al., “Ensuring the Biological Safety of Plasma-Derived Therapeutic Proteins: Detection, Inactivation, and Removal of Pathogens,” BioDrugs 19(2):79-96 (2005). In some cases, fractionation comprises slowly thawing frozen plasma (e.g., at about 2° C.-4° C.) and centrifuging to remove cryoprecipitate prior to altering the solubility of the proteins remaining in the supernatant by manipulating, e.g., concentration of ethanol, temperature, pH, and/or ionic strength. In some cases, fractionation further comprises centrifugation, filtration (e.g., nanofiltration), and/or pathogen reduction treatments (see, e.g., Cai et al.).

In some cases, the pooled IgG antibodies pooled human plasma, pooled human serum, cryoprecipitate depleted plasma, pooled human serum or plasma that has been ethanol precipitated, human Cohn II/III plasma fraction, or human Cohn plasma I/II/III fraction. In some cases, the pooled IgG antibodies are human Cohn II/III plasma fraction, or human Cohn plasma I/II/III fraction.

In some cases, the human Cohn II/III plasma fraction is obtained by a process comprising: (a) cryoseparation; (b); alcohol precipitation; and (c) collecting the precipitate, thereby obtaining human Cohn I plasma fraction. In some cases, alcohol precipitation is ethanol precipitation, e.g., cold ethanol precipitation, e.g., ethanol precipitation from about −3° C. to about −5° C. In some cases, the alcohol precipitation comprises alcohol precipitation at or at about 8% (v/v) alcohol, e.g., at or at about 8% (v/v) ethanol. In some cases, alcohol precipitation is carried out at or at about pH 7.2. In some cases, the human Cohn II/III plasma fraction is obtained by a process comprising: (a) providing human Cohn I plasma fraction supernatant; (b) a second alcohol precipitation; and (c) collecting the precipitate, thereby obtaining human Cohn II/III plasma fraction. In some cases, the second alcohol precipitation comprises alcohol precipitation at or at about 20% (v/v) alcohol, e.g., at or at about 20% to or to about 25% (v/v) ethanol. In some cases, the second alcohol precipitation is carried out at or at about pH 6.9. In some cases, the human Cohn I/II/III fraction is obtained by combining human Cohn I plasma fraction and human Cohn II/III plasma fraction.

In some embodiments, the methods described herein include providing a mixture of IgG antibodies. In some embodiments, providing a mixture of IgG antibodies includes (a) providing pooled plasma from at least 1000 human subjects; and (b) isolating a mixture of IgG antibodies from the pooled plasma. In some embodiments, the mixture of IgG antibodies are isolated from intravenous immunoglobulin. In some embodiments, the mixture of IgG antibodies are intravenous immunoglobulin.

In some embodiments, isolating a mixture of IgG antibodies from the pooled plasma comprises precipitation (e.g., caprylate precipitation and/or polyethylene glycol (PEG) precipitation), chromatography (e.g., ion exchange chromatography, hydrophilic interaction chromatography, e.g., hydrophilic interaction liquid chromatography, and/or high performance liquid chromatography, e.g., C18 high performance liquid chromatography), filtration, delipidation, pathogen inactivation, e.g., viral inactivation (e.g., detergent treatment and/or caprylate precipitation), or a combination thereof.

In some embodiments, the step of isolating a mixture of IgG antibodies from the pooled plasma comprises ethanol precipitation and/or caprylic acid (also called octanoic acid) precipitation.

In some embodiments, the step of isolating a mixture of IgG antibodies from the pooled plasma comprises PEG-4000 and/or PEG-6000 precipitation.

In some embodiments, the step of isolating a mixture of IgG antibodies from the pooled plasma comprises ethanol precipitation or caprylic acid precipitation followed by PEG-4000 and/or PEG-6000 precipitation, e.g., caprylic acid precipitation followed by PEG-4000 precipitation.

In some embodiments, the step of isolating a mixture of IgG antibodies from the pooled plasma comprises binding IgG antibodies to an ion exchange column and eluting the IgG antibodies from an ion exchange column.

In some embodiments, the step of isolating a mixture of IgG antibodies from the pooled plasma comprises adding one or more of: a delipidation agent, a high purity diatomite filter media, or a fumed silica to the mixture of IgG antibodies. In some embodiments, the delipidation agent, high purity diatomite filter media, or fumed silica is added to the mixture of IgG antibodies prior to buffer exchange.

In some cases, isolating a mixture of IgG antibodies, e.g., the Cohn I/II/III fraction or Cohn II/III fraction, comprises reducing the amount of non-IgG protein. In some cases, isolating a mixture of IgG antibodies, e.g., the Cohn I/II/III fraction or Cohn II/III fraction, comprises reducing the amount of non-protein components. In some cases, isolating a mixture of IgG antibodies, e.g., the Cohn I/II/III fraction or Cohn II/III fraction, comprises viral inactivation.

Purification techniques suitable for reducing the amount of non-IgG proteins and/or non-protein components are known and described in the art. See, e.g., Ofosu et al., “Plasma-Derived Biological Medicines Used to Promote Haemostasis,” Thromb Haemost 99:851-62 (2008).

Suitable viral inactivation methods are known and described in the art. See, e.g., Cai et al., “Ensuring the Biological Safety of Plasma-Derived Therapeutic Proteins: Detection, Inactivation, and Removal of Pathogens,” BioDrugs 19(2):79-96 (2005).

### Enzymatic Galactosylation and Sialylation

Enzymatic galactosylation and sialylation reactions suitable for the methods described herein are described, for example, in PCT/US2021/033150.

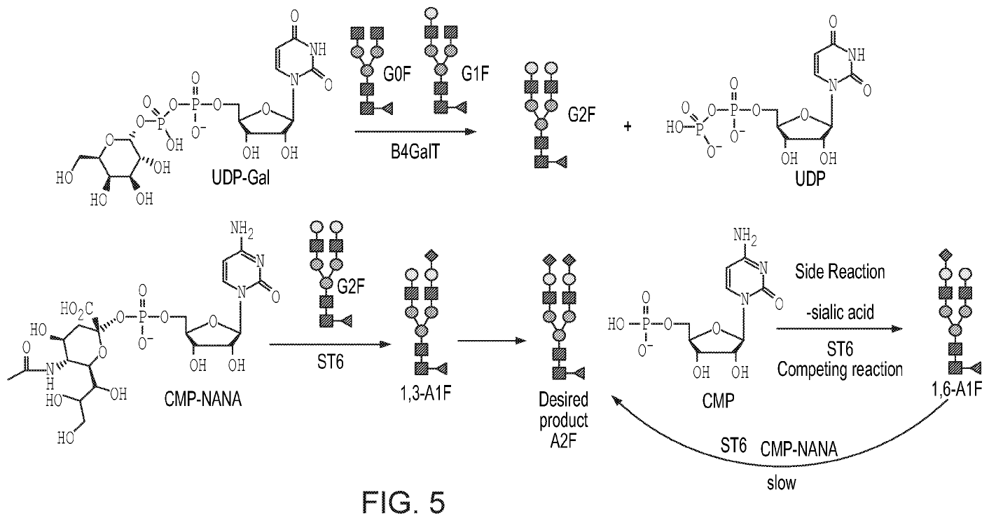

The methods described herein can comprise a galactosylation step. An exemplary galactosylation reaction is depicted in FIG. 5. Thus, provided herein is a method for galactosylating antibod(ies), e.g., antibod(ies) described herein, by providing a composition (a galactosylation mixture) comprising: antibod(ies), e.g., antibod(ies) described herein; a galactosylating enzyme, e.g., a galactosylating enzyme described herein, e.g., B4GalT or enzymatically active portion of variant thereof; UDP-gal or salt thereof; and incubating the composition under conditions effective for galactosylating the antibody, e.g., as described herein, thereby producing galactosylated antibod(ies).

The methods described herein can comprise a sialylation step. An exemplary sialylation reaction is depicted in FIG. 5. Thus, provided herein is a method for sialylating, e.g., hyper-sialylating, antibod(ies), e.g., antibod(ies) described herein, by providing a composition (a sialylation reaction mixture) comprising: galactosylated antibod(ies), e.g., as described herein; a sialylating enzyme, e.g., a sialylating enzyme described herein, e.g., ST6Gal1 or enzymatically active portion or variant thereof; CMP-NANA or a salt thereof; and incubating the composition under conditions effective for sialylating the antibod(ies), e.g., as described herein.

In some embodiments, the galactosylation step and the sialylation step are carried out sequentially in the same reaction mixture, that is, the galactosylation reaction mixture becomes the sialylation reaction mixture upon addition of the sialylating enzyme and CMP-NANA or salt thereof. In some embodiments, the galactosylation reaction mixture is not filtered, fractionated, or purified prior to the sialylation step. In some embodiments, the galactosylation step and the sialylation step are carried out separately, e.g., pre-galactosylated antibod(ies) are provided, though they may have been processed (e.g., filtered, fractionated, or purified) and/or stored prior to the sialylation step.

Thus, the methods described herein can also comprise a sequential galactosylation and sialylation step. An exemplary galactosylation and sialylation reaction is depicted in FIG. 5. Thus, provided herein is a method for galactosylating and sialylating, e.g., hyper-sialylating, antibod(ies), e.g., antibod(ies) described herein, by a) providing a composition (a galactosylation reaction mixture) comprising: antibod(ies), e.g., as described herein; a galactosylating enzyme, e.g., a galactosylating enzyme described herein, e.g., B4GalT or enzymatically active portion or variant thereof; UDP-gal or a salt thereof; and b) incubating the composition under conditions effective for galactosylating the antibod(ies), e.g., as described herein; c) adding a sialylating enzyme, e.g., a sialylating enzyme described herein, e.g., ST6Gal1 or enzymatically active portion or variant thereof and CMP-NANA or salt thereof to the galactosylation reaction mixture, thereby producing a sialylation reaction mixture; and d) incubating the composition under conditions effective for sialylating the galactosylated antibod(ies), e.g., as described herein.

Also provided herein is a method for galactosylating and sialylating, e.g., hyper-sialylating antibod(ies), e.g., antibod(ies) described herein, by providing a composition comprising: antibod(ies), e.g., as described herein; a galactosylating enzyme, e.g., a galactosylating enzyme described herein, e.g., B4GalT or enzymatically active portion or variant thereof; UDP-gal or a salt thereof; a sialylating enzyme, e.g., a sialylating enzyme described herein, e.g., ST6Gal1 or enzymatically active portion or variant thereof; CMP-NANA or salt thereof; and d) incubating the composition under conditions effective for galactosylating and sialylating the antibod(ies), e.g., as described herein.

In some embodiments, the galactosylation reaction mixture and/or the sialylation reaction mixture comprises Bis (2-hydroxyethyl) aminotris (hydroxymethyl)methane (BIS-TRIS) buffer.

In some embodiments, the galactosylation reaction mixture and/or the sialylation reaction mixture comprises MnCl₂.

In some embodiments, one or more component(s) of one or more of the reaction mixture(s) are supplemented during the incubation. That is, the reaction mixture may comprise an amount of the component at the beginning of the reaction (which may change during the course of the reaction), but also be supplemented with additional amounts of the component(s) during the reaction.

In some embodiments, the B4GalT comprises or consists of an amino acid sequence is at least 90% identical SEQ ID NO: 12 or SEQ ID NO: 13.

In some embodiments, the ST6Gal1 comprises or consists of an amino acid sequence that is at least 90% identical SEQ ID NO: 19 or SEQ ID NO: 21.

In some embodiments, at least or about 60%, 65%, 70%, 75%, 80%, or 85% of the branched glycans on the sialylated antibod(ies), e.g., hsIgG, have a sialic acid on both the α1,3 branch and the α1,6 branch.

In some embodiments, about or at least 60%, 65%, 70%, 75%, 80%, or 85% of the branched Fc glycans on the sialylated antibod(ies), e.g., hsIgG, have a sialic acid on both the α1,3 branch and the α1,6 branch.

In some embodiments, about or at least 60%, 65%, 70%, 75%, 80%, or 85% of the branched glycans on the Fab domain of the sialylated antibod(ies), e.g., hsIgG, have a sialic acid on both the α 1,3 arm and the a 1,6 arm that is connected through a NeuAc-α 2,6-Gal terminal linkage.

In some embodiments, about or at least 80% of the branched Fc glycans on the sialylated antibod(ies), e.g., hsIgG, have a sialic acid on both the α1,3 branch and the α1,6 branch.

In some embodiments, about or at least 60%, 65%, 70% of the branched glycans on the Fab domain of the sialylated antibod(ies), e.g., hsIgG, have a sialic acid on both the α 1,3 arm and the a 1,6 arm that is connected through a NeuAc-α 2,6-Gal terminal linkage.

In some embodiments, about or at least 85% of the of the branched Fc glycans on the sialylated antibod(ies), e.g., hsIgG, have a sialic acid on both the α1,3 branch and the α1,6 branch.

In some embodiments, about or at least 60%, 65%, 70% of the branched glycans on the Fab domain of the sialylated antibod(ies), e.g., hsIgG, have a sialic acid on both the α 1,3 arm and the a 1,6 arm that is connected through a NeuAc-α 2,6-Gal terminal linkage.

In some embodiments, about or at least 90% of the of the branched Fc glycans on the sialylated antibod(ies), e.g., hsIgG, have a sialic acid on both the α1,3 branch and the α1,6 branch.

In some embodiments, about or at least 60%, 65%, 70% of the branched glycans on the Fab domain of the sialylated antibod(ies), e.g., hsIgG, have a sialic acid on both the α 1,3 arm and the a 1,6 arm that is connected through a NeuAc-α 2,6-Gal terminal linkage.

In some cases, the hsIgG can be purified, e.g., as described in PCT/US2021/033156.

An exemplary galactosylation and sialylation reaction is shown in the table below.

### Buffers

Suitable buffers for use in the compositions and methods described herein include, but are not limited to those shown in Table 9. In some cases, the buffer is present in the composition or reaction mixture at 10-500 mM, e.g., 10-100 mM, e.g., about 50 mM. In some cases, the buffer has a pH of 5.5-8.5, e.g., 6.5-7.5, e.g., about 7.0.

TABLE 9
Buffers

| Buffer | Buffer Name | Structure |
|---|---|---|
| MOPS | 3-(N- morpholino) propanesulfonic acid | 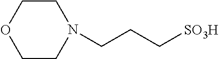 [SMILES: O=S(=O)(O)CCCN1CCOCC1] |
| MES | 2-(N- morpholino)ethanesulfonic acid | 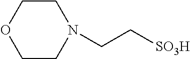 [SMILES: O=S(=O)(O)CCN1CCOCC1] |
| BIS-TRIS | Bis(2- hydroxyethyl)aminoOtris (hydroxymethyl)methane | 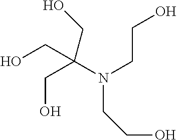 [SMILES: OCCN(CCO)C(CO)(CO)CO] |
| PIPES | 1,4- Piperazinediethanesulfonic acid | 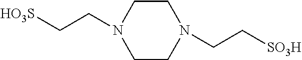 [SMILES: O=S(=O)(O)CCN1CCN(CCS(=O)(=O)O)CC1] |
| BES | N,N-Bis(2-hydroxyethyl)-2- aminoethanesulfonic acid | 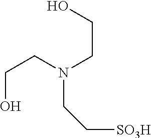 [SMILES: O=S(=O)(O)CCN(CCO)CCO] |
| MOPSO | 3-morpholino-2- hydroxypropanesulfonic acid | 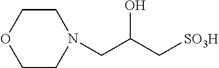 [SMILES: O=S(=O)(O)CC(O)CN1CCOCC1] |
| TEA | Triethanolamine | 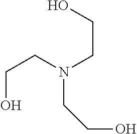 |
| POPSO | Piperazine-N-N′-bis(2- hydroxypropanesulfonic acid | 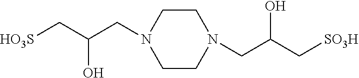 [SMILES: O=S(=O)(O)CC(O)CN1CCN(CC(O)CS(=O)(=O)O)CC1] |
| EPPS | 4-(2-Hydroxyethyl)-1- piperazinepropanesulfonic acid | 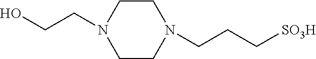 [SMILES: O=S(=O)(O)CCCN1CCN(CCO)CC1] |

### Evaluation of Glycans

As described above, immunoglobulins, e.g., IgG antibodies, can be sialylated in vitro by carrying out a galactosylation step followed by a sialylation step. Beta-1,4-galactosyltransferase 1 (B4GalT) is a Type II Golgi membrane-bound glycoprotein that transfers galactose from uridine 5′-diphosphosegalactose (UDP-Gal) to GlcNAc as a β-1,4 linkage. Alpha-2,6-sialyltransferase 1 (also referred to ST6 or ST6Gal) is a Type II Golgi membrane-bound glycoprotein that transfers sialic acid from cytidine 5′-monophospho-N-acetylneuraminic acid (CMP-NANA) to Gal as an α-2,6 linkage. Schematically, the reactions proceed as shown in FIG. 5.

Glycans of polypeptides can be evaluated using any methods known in the art. For example, sialylation of glycan compositions (e.g., level of branched glycans that are sialylated on an α1,3 branch and/or an α1,6 branch) can be characterized using methods described in WO2014/179601.

In some embodiments of the hsIgG compositions prepared by the methods described herein, at least 50%, 55%, 60%, 65%, 70%, 75%, 80%, 85%, or 90% of the branched glycans on the Fc domain have a sialic acid on both the α 1,3 arm and the a 1,6 arm that is connected through a NeuAc-α 2,6-Gal terminal linkage. In addition, in some embodiments, at least 40%, 50%, 60%, 65%, 70%, 75%, 80%, or 85% of the branched glycans on the Fab domain have a sialic acid on both the α 1,3 arm and the a 1,6 arm that is connected through a NeuAc-α 2,6-Gal terminal linkage. Overall, in some embodiments, at least 50%, 55%,60%, 65%, 70%, 75%, 80%, 85%, or 90% of the branched glycans have a sialic acid on both the α 1,3 arm and the a 1,6 arm that is connected through a NeuAc-α 2,6-Gal terminal linkage.

Glycans of glycoproteins can be evaluated using any methods known in the art. For example, sialylation of glycan compositions (e.g., level of branched glycans that are sialylated on an α1,3 arm and/or an α1,6 arm) can be characterized using methods described in, e.g., Barb, Biochemistry 48:9705-9707 (2009); Anumula, J. Immunol. Methods 382:167-176 (2012); Gilar et al., Analytical Biochem. 417:80-88 (2011); Wuhrer et al., J. Chromatogr. B. 849:115-128 (2007). In some embodiments, in addition to evaluation of sialylation of glycans, one or more parameters described in Table 9 are evaluated.

In some instances, glycan structure and composition as described herein are analyzed, for example, by one or more, enzymatic, chromatographic, mass spectrometry (MS), chromatographic followed by MS, electrophoretic methods, electrophoretic methods followed by MS, nuclear magnetic resonance (NMR) methods, and combinations thereof. Exemplary enzymatic methods include contacting a glycoprotein preparation with one or more enzymes under conditions and for a time sufficient to release one or more glycan(s) (e.g., one or more exposed glycan(s)). In some instances, the one or more enzymes include(s) PNGase F. Exemplary chromatographic methods include, but are not limited to, Strong Anion Exchange chromatography using Pulsed Amperometric Detection (SAX-PAD), liquid chromatography (LC), high performance liquid chromatography (HPLC), ultra performance liquid chromatography (UPLC), thin layer chromatography (TLC), amide column chromatography, and combinations thereof. Exemplary mass spectrometry (MS) include, but are not limited to, tandem MS, LC-MS, LC-MS/MS, matrix assisted laser desorption ionization mass spectrometry (MALDI-MS), Fourier transform mass spectrometry (FTMS), ion mobility separation with mass spectrometry (IMS-MS), electron transfer dissociation (ETD-MS), and combinations thereof. Exemplary electrophoretic methods include, but are not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof. Exemplary nuclear magnetic resonance (NMR) include, but are not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear Overhauser effect spectroscopy NMR (ROESY-NMR), nuclear Overhauser effect spectroscopy (NOESY-NMR), and combinations thereof.

In some instances, techniques described herein may be combined with one or more other technologies for the detection, analysis, and or isolation of glycans or glycoproteins. For example, in certain instances, glycans are analyzed in accordance with the present disclosure using one or more available methods (to give but a few examples, see Anumula, Anal. Biochem., 350(1):1, 2006; Klein et al., Anal. Biochem., 179:162, 1989; and/or Townsend, R.R. Carbohydrate Analysis” High Performance Liquid Chromatography and Capillary Electrophoresis., Ed. Z. El Rassi, pp 181-209, 1995; WO2008/128216; WO2008/128220; WO2008/128218; WO2008/130926; WO2008/128225; WO2008/130924; WO2008/128221; WO2008/128228; WO2008/128227; WO2008/128230; WO2008/128219; WO2008/128222; WO2010/071817; WO2010/071824; WO2010/085251; WO2011/069056; and WO2011/127322). For example, in some instances, glycans are characterized using one or more of chromatographic methods, electrophoretic methods, nuclear magnetic resonance methods, and combinations thereof. In some instances, methods for evaluating one or more target protein specific parameters, e.g., in a glycoprotein preparation, e.g., one or more of the parameters disclosed herein, can be performed by one or more of following methods.

In some instances, methods for evaluating one or more target protein specific parameters, e.g., in a glycoprotein preparation, e.g., one or more of the parameters disclosed herein, can be performed by one or more of following methods.

TABLE 10
Exemplary methods of evaluating parameters:

| Method(s) | Relevant Literature | Parameter |
|---|---|---|
| C18 UPLC Mass | Chen and Flynn, Anal. | Glycan(s) |
| Spec.* | Biochem., 370: 147-161 | (e.g., N-linked glycan, |
| | (2007) | exposed N-linked glycan, |
| | Chen and Flynn, J. Am. | glycan detection, glycan |
| | Soc. Mass Spectrom., | identification, and |
| | 20: 1821-1833 (2009) | characterization; site |
| | | specific glycation; |
| | | glycoform detection (e.g., |
| | | parameters 1-7); percent |
| | | glycosylation; and/or |
| | | aglycosyl) |
| Peptide LC-MS | Dick et al., Biotechnol. | C-terminal lysine |
| (reducing/non- | Bioeng., 100: 1132-1143 | |
| reducing) | (2008) | |
| | Yan et al., J. Chrom. A., | |
| | 1164: 153-161 (2007) | |
| | Chelius et al., Anal. | |
| | Chem., 78: 2370-2376 | |
| | (2006) | |
| | Miller et al., J. Pharm. | |
| | Sci., 100: 2543-2550 | |
| | (2011) | |
| LC-MS (reducing/non- | Dick et al., Biotechnol. | C-terminal lysine |
| reducing/alkylated) | Bioeng., 100: 1132-1143 | |
| | (2008) | |
| | Goetze et al., Glycobiol., | |
| | 21: 949-959 (2011) | |
| Weak cation exchange | Dick et al., Biotechnol. | C-terminal lysine |
| (WCX) | Bioeng., 100: 1132-1143 | |
| chromatography | (2008) | |
| LC-MS (reducing/non- | Dick et al., Biotechnol. | N-terminal pyroglu |
| reducing/alkylated) | Bioeng., 100: 1132-1143 | |
| | (2008) | |
| | Goetze et al., Glycobiol., | |
| | 21: 949-959 (2011) | |
| PeptideLC-MS | Yan et al., J. Chrom. A., | N-terminal pyroglu |
| (reducing/non- | 1164: 153-161 (2007) | |
| reducing) | Chelius et al., Anal. | |
| | Chem., 78: 2370-2376 | |
| | (2006) | |
| | Miller et al., J. Pharm. | |
| | Sci., 100: 2543-2550 | |
| | (2011) | |
| Peptide LC-MS | Yan et al., J. Chrom. A., | Methionine oxidation |
| (reducing/non- | 1164: 153-161 (2007); | |
| reducing) | Xie et al., mAbs, 2: 379- | |
| | 394 (2010) | |
| Peptide LC-MS | Miller et al., J. Pharm. | Site specific glycation |
| (reducing/non- | Sci., 100: 2543-2550 | |
| reducing) | (2011) | |
| Peptide LC-MS | Wang et al., Anal. Chem., | Free cysteine |
| (reducing/non- | 83: 3133-3140 (2011); | |
| reducing) | Chumsae et al., Anal. | |
| | Chem., 81: 6449-6457 | |
| | (2009) | |
| Bioanalyzer | Forrer et al., Anal. | Glycan (e.g., N-linked |
| (reducing/non- | Biochem., 334: 81-88 | glycan, exposed N-linked |
| reducing)* | (2004) | glycan) |
| | | (including, for example, |
| | | glycan detection, |
| | | identification, and |
| | | characterization; site |
| | | specific glycation; |
| | | glycoform detection; percent |
| | | glycosylation; and/or |
| | | aglycosyl) |
| LC-MS (reducing/non- | Dick et al., Biotechnol. | Glycan (e.g., N-linked |
| reducing/alkylated)* | Bioeng., 100: 1132-1143 | glycan, exposed N-linked |
| *Methods include | (2008) | glycan) |
| removal (e.g., | Goetze et al., Glycobiol., | (including, for example, |
| enzymatic, chemical, | 21: 949-959 (2011) | glycan detection, |
| and physical) of | Xie et al., mAbs, 2: 379- | identification, and |
| glycans | 394 (2010) | characterization; site |
| | | specific glycation; |
| | | glycoform detection; percent |
| | | glycosylation; and/or |
| | | aglycosyl) |
| Bioanalyzer | Forrer et al., Anal. | Light chain: Heavy chain |
| (reducing/non- | Biochem., 334: 81-88 | |
| reducing) | (2004) | |
| Peptide LC-MS | Yan et al., J. Chrom. A., | Non-glycosylation-related |
| (reducing/non- | 1164: 153-161 (2007) | peptide modifications |
| reducing) | Chelius et al., Anal. | (including, for example, |
| | Chem., 78: 2370-2376 | sequence analysis and |
| | (2006) | identification of sequence |
| | Miller et al., J. Pharm. | variants; oxidation; |
| | Sci., 100: 2543-2550 | succinimide; aspartic acid; |
| | (2011) | and/or site-specific aspartic |
| | | acid) |
| Weak cation exchange | Dick et al., Biotechnol. | Isoforms (including, for |
| (WCX) | Bioeng., 100: 1132-1143 | example, charge variants |
| chromatography | (2008) | (acidic variants and basic |
| | | variants); and/or deamidated |
| | | variants) |
| Anion-exchange | Ahn et al., J. Chrom. B, | Sialylated glycan |
| chromatography | 878: 403-408 (2010) | |
| Anion-exchange | Ahn et al., J. Chrom. B, | Sulfated glycan |
| chromatography | 878: 403-408 (2010) | |
| 1,2-diamino-4,5- | Hokke et al., FEBS Lett., | Sialic acid |
| methylenedioxybenzene | 275: 9-14 (1990) | |
| (DMB) labeling method | | |
| LC-MS | Johnson et al., Anal. | C-terminal amidation |
| | Biochem., 360: 75-83 | |
| | (2007) | |
| LC-MS | Johnson et al., Anal. | N-terminal fragmentation |
| | Biochem., 360: 75-83 | |
| | (2007) | |
| Circular dichroism | Harn et al., Current | Secondary structure |
| spectroscopy | Trends in Monoclonal | (including, for example, |
| | Antibody Development | alpha helix content and/or |
| | and Manufacturing, S. J. | beta sheet content) |
| | Shire et al., eds, 229-246 | |
| | (2010) | |
| Intrinsic and/or ANS | Harn et al., Current | Tertiary structure |
| dye fluorescence | Trends in Monoclonal | (including, for example, |
| | Antibody Development | extent of protein folding) |
| | and Manufacturing, S. J. | |
| | Shire et al., eds, 229-246 | |
| | (2010) | |
| Hydrogen-deuterium | Houde et al., Anal. | Tertiary structure and |
| exchange-MS | Chem., 81: 2644-2651 | dynamics (including, for |
| | (2009) | example, accessibility f |
| | | amide protons to solvent |
| | | water) |
| Size-exclusion | Carpenter et al., J. Pharm. | Extent of aggregation |
| chromatography | Sci., 99: 2200-2208 (2010) | |
| Analytical | Pekar and Sukumar, Anal. | |
| ultracentrifugation | Biochem., 367: 225-237 | |
| | (2007) | |

The literature recited above are hereby incorporated by reference in their entirety or, in the alternative, to the extent that they pertain to one or more of the methods for determining a parameter described herein.

### Pharmaceutical Compositions and Administration

Hypersialylated IgG can be incorporated into a pharmaceutical composition. For example, the pharmaceutical composition can be formulated by suitably combining the hsIgG with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in the pharmaceutical preparations is such that a suitable dose within the designated range is provided.

The hsIgG can be formulated for intravenous administration.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

A suitable means of administration can be selected based on the age and condition of the patient. A suitable dose of hsIgG prepared by the methods described herein can be about the same or less than (e.g., 20%, 35%, 40%, 50%, 60%, 70% or 80% less) the suitable or approved dose of commercially available IVIg preparations. The dose and method of administration varies depending on the weight, age, condition, and the like of the patient, and can be suitably selected as needed by those skilled in the art.

### EXAMPLES

The invention is further described in the following examples, which do not limit the scope of the invention described in the claims.

### Example 1: Hypersialylation of IgG from Cohn fraction

IgG in which more than 50% of the overall branched glycans are disialylated can be prepared as follows.

Briefly, a mixture of IgG antibodies (e.g., from Cohn II/III or Cohn I/II/III) is exposed to an enzymatic reaction using β1,4 galactosyltransferase 1 (B4GalT) and α2,6-sialyltransferase (ST6Gal1) enzymes. The B4GalT does not need to be removed from the reaction before addition of ST6Gal1 and no partial or complete purification of the product is needed between the enzymatic reactions. Therefore the reaction may be sequential or concurrent.

The galactosyltransferase enzyme selectively adds galactose residues to pre-existing asparagine-linked glycans. The resulting galactosylated glycans serve as substrates for the sialic acid transferase enzyme which selectively adds sialic acid residues to cap the asparagine-linked glycan structures attached to the protein. Thus, for disialylation the overall sialylation reaction employed two sugar nucleotides (uridine 5′-diphosphogalactose (UDP-Gal) and cytidine-5′-monophospho-N-acetylneuraminic acid (CMP-NANA)). The latter can be replenished periodically to increase disialylated product relative to monosialylated product. The reaction includes the co-factor manganese (II) chloride.

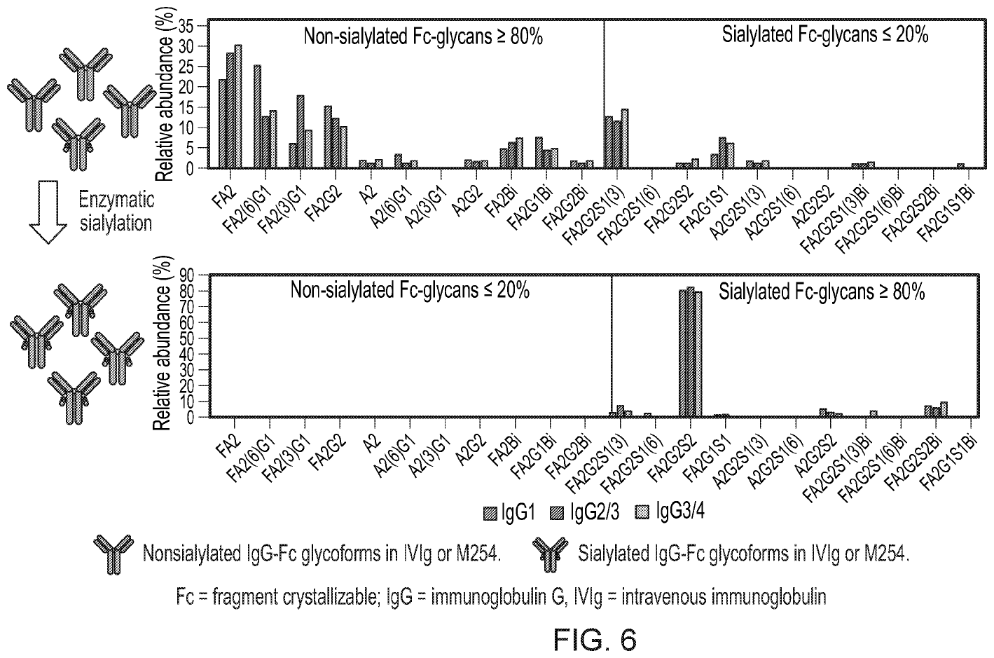

A representative example of the IgG-Fc glycan profile for such a reaction starting with IVIg and the reaction product is shown in FIG. 6. The left panel is a schematic representation of enzymatic sialylation reaction to transform IgG to hsIgG; the right panel is the IgG Fc glycan profile for the starting IVIg and hsIgG. In this study, glycan profiles for the different IgG subclasses are derived via glycopeptide mass spectrometry analysis. The peptide sequences used to quantify glycopeptides for different IgG subclasses were: IgG1=EEQYNSTYR (SEQ ID NO: 1), IgG2/3=EEQFNSTFR (SEQ ID NO: 2, IgG3/4=EEQYNSTFR (SEQ ID NO: 3) and EEQFNSTYR (SEQ ID NO: 4).

The glycan data is shown per IgG subclass. Glycans from IgG3 and IgG4 subclasses cannot be quantified separately. As shown, for IVIg the sum of all the nonsialylated glycans is more than 80% and the sum of all sialylated glycans is <20% (top graph). For the reaction product, the sum for all nonsialylated glycans is <20% and the sum for all sialylated glycans is more than 80% (bottom graph). Nomenclature for different glycans listed in the glycoprofile use the Oxford notation for N linked glycans.

The starting material for the following examples was a lyophilized solid of Cohn II,III sourced from Sigma Aldrich (G2388; 78% protein by weight, primarily γ-globulin and β-globulin). A portion of the material was not completely soluble in the reaction buffer of choice (BIS-TRIS) nor at the concentration of choice (>100 mg/mL). Insoluble materials were generally gel-like rather than solid precipitates, possibly due to the presence of lipoproteins.

Five different preparations of the Cohn II/III material were generated (Preparations A-E) and used to prepare hsIgG. Concentrations were estimated by A280 using the extinction coefficient for IgG (1.37). It should be noted that the IgG concentration was assessed by absorption at A280, but this signal likely reflects the presence of non-IgG protein as well as non-protein material. Thus, the actual IgG concentration may be lower than indicated.

Preparation A: Cohn fraction II,III dissolved in 50 mM pH 6.9 BIS-TRIS buffer to a measured (A280) concentration of 109 mg/mL. Undissolved material was allowed to settle.

Preparation B: Cohn fraction II,III dissolved in 50 mM pH 6.9 BIS-TRIS buffer to a measured (A280) concentration of 97 mg/mL. Undissolved material was allowed to settle.

Preparation C: Cohn fraction II,III (640 mg) dissolved in 30 mL 50 mM pH 6.9 BIS-TRIS buffer (21.3 mg/ml), sterile filtered (0.2 μm) to remove undissolved material and then then buffer exchanged into 50 mM pH 6.9 BIS-TRIS buffer using a G25 desalting column. The resulting material was allowed to stand, the liquid was decanted away from the settled solids, and the liquid was concentrated to 50.6 mg/mL using 10 kDa MWCO Vivaspin Turbo 15 devices.

Preparation D: Cohn fraction II,III dissolved in 50 mM pH 6.9 BIS-TRIS buffer, buffer exchanged into 50 mM pH 6.9 BIS-TRIS buffer using a 10 kDa G2 dialysis cassette. Undissolved material was allowed to settle and the supernatant was decanted off. Concentration in the supernatant was 37 mg/mL.

Preparation E: Cohn fraction II,III material (622 mg) dissolved in 9 mL 50 mM pH 6.9 BIS-TRIS buffer (691 mg/ml) and buffer exchanged into 50 mM pH 6.9 BIS-TRIS buffer using a 10 kDa G2 dialysis cassette over 54 h with one change in dialysis buffer. The material was centrifuged to settle undissolved material and the supernatant was decanted off. Concentration in supernatant was 38 mg/mL.

Galactosylation using human B4GalT enzyme, UDP-Gal sugar nucleotide donor, and manganese(II) chloride (MnCl₂) in BIS-TRIS pH 6.9 buffer was done first for 18-22h at 37° C. This was followed by sialylation with addition of a deletion mutant of human ST6 sialyltransferase that includes amino acids 109-406 of SEQ ID NO: 14 (SEQ ID NO: 20) and half of the total amount of the CMP-NANA sugar donor. The second half of the CMP-NANA was added at −9 h post sialylation start. Total sialylation time was 30-32 h. The reaction scale was 20 mg protein. The amounts of reagents were varied across the reactions.

The extent of Fc galactosylation and sialylation was determined by glycopeptide analysis as follows. A 50 μg sample was denatured, reduced, and alkylated. The resulting material was subjected to trypsin digestion and analysis by LCMS. The following glycans on the Fc glycopeptide were quantified.

Glycoform
G0F
G1F
G2F
A1F
A2F
G1F + NeuAc
G0F + bisecting_GlcNAc
G1F + bisecting_GlcNAc
G2F + bisecting_GlcNAc
A1F + bisecting_GlcNAc
A2F + bisecting_GlcNAc
G0
G1
G2
A1
A2
G1 + NeuAc

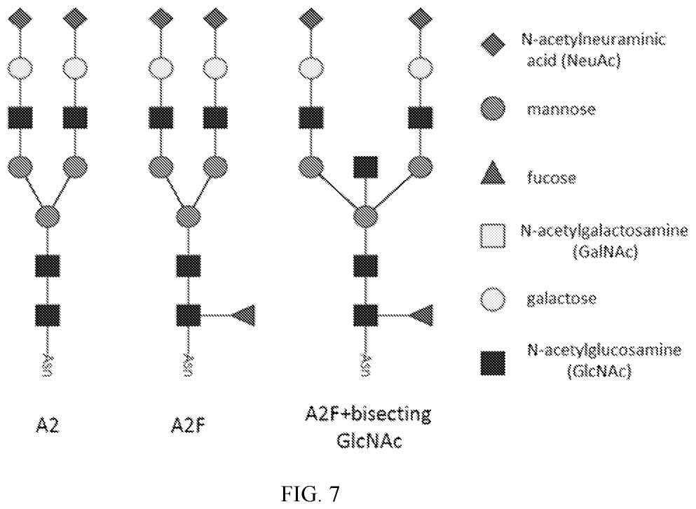

Total sialylation was defined as the sum of the LCMS peak areas for the glycans which were fully galactosylated and sialylated (A2, A2F, and A2F+bisecting GlcNAc) (FIG. 7).

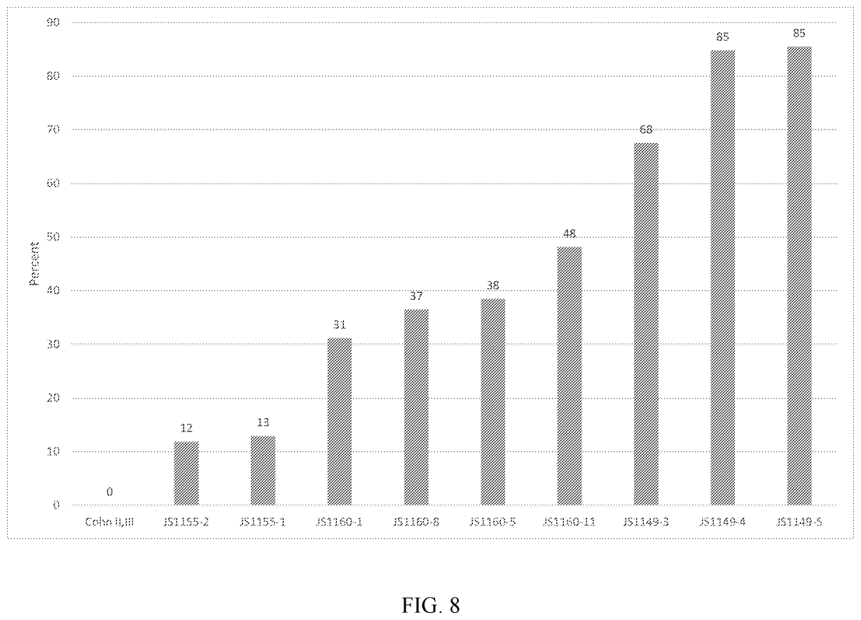

A first set of reactions was run on Cohn II/III fraction material directly dissolved in BIS-TRIS buffer using the conditions in Table 11. FIGS. 8, 10, and 11 show the fraction of branched glycans that are disialylated for each reaction.

TABLE 11
Conditions for first set of reactions

| | | B4GalT | ST6 | UDP-Gal | CMP-NANA | |
|---|---|---|---|---|---|---|
| | | (mU/mg | (mU/mg | (nmol/mg | (nmol/mg | MnCl2 |
| Reaction | Material | protein) | protein) | protein) | protein) | (mM) |
| JS1155-2 | B (37 mg/mL) | 9.3 | 19 | 37.5 | 220 | 2.4 |
| JS1155-1 | B (97 mg/mL) | 9.3 | 19 | 37.5 | 220 | 5.3 |
| JS1160-1 | B (97 mg/mL) | 14.8 | 31.1 | 38 | 220 | 7.4 |
| JS1160-8 | B (97 mg/mL) | 15.2 | 30.9 | 95 | 445 | 7.4 |
| JS1160-5 | B (97 mg/mL) | 22.5 | 46.9 | 37.5 | 220 | 7.4 |
| JS1160-11 | B (97 mg/mL) | 22.5 | 46.9 | 95 | 445 | 7.4 |
| JS1149-3 | A (109 mg/mL) | 93 | 201 | 380 | 2200 | 58 |
| JS1149-4 | B (97 mg/mL) | 93 | 201 | 380 | 2200 | 9.3 |
| JS1149-5 | B (37 mg/mL) | 93 | 201 | 380 | 2200 | 9.2 |

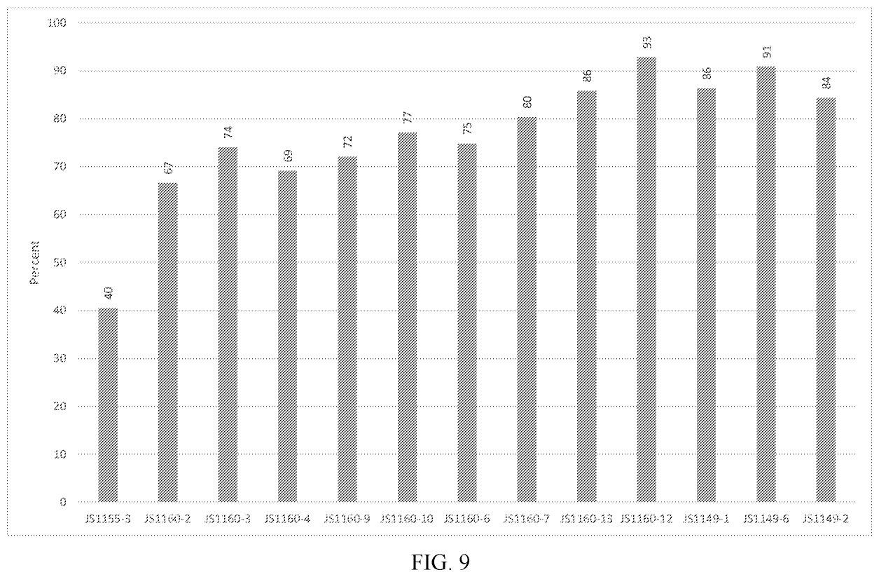

A second set of reactions was run on Cohn II/III fraction material buffer exchanged into BIS-TRIS buffer using the conditions in Table 12. FIGS. 9, 12, and 13 show the fraction of branched glycans that are disialylated for each reaction.

TABLE 12
Conditions for second set of reactions

| | | B4GalT | ST6 | UDP-Gal | CMP-NANA | |
|---|---|---|---|---|---|---|
| | | (mU/mg | (mU/mg | (nmol/mg | (nmol/ | MnCl2 |
| Reaction | Material | protein) | protein) | protein) | protein) | (mM) |
| JS1155-3 | D (37 mg/mL) | 9.3 | 19 | 37.5 | 220 | 2.3 |
| JS1160-2 | C (37 mg/mL) | 14.8 | 31.1 | 38 | 220 | 7.4 |
| JS1160-3 | E (38 mg/mL) | 14.8 | 31.1 | 38 | 220 | 7.5 |
| JS1160-4 | D (37 mg/mL) | 14.8 | 31.1 | 38 | 220 | 7.4 |
| JS1160-9 | C (37 mg/mL) | 15.2 | 30.9 | 95 | 445 | 7.4 |
| JS1160-10 | E (38 mg/mL) | 15.2 | 30.9 | 95 | 445 | 7.5 |
| JS1160-6 | C (37 mg/mL) | 22.5 | 46.9 | 37.5 | 220 | 7.4 |
| JS1160-7 | E (38 mg/mL) | 22.5 | 46.9 | 37.5 | 220 | 7.5 |
| JS1160-13 | E (38 mg/mL) | 22.5 | 46.9 | 95 | 445 | 7.5 |
| JS1160-12 | C (37 mg/mL) | 22.5 | 93.8 | 95 | 700 | 7.4 |
| JS1149-1 | D (37 mg/mL) | 37 | 81 | 152 | 880 | 15.7 |
| JS1149-6 | D (37 mg/mL) | 93 | 201 | 380 | 2200 | 9.0 |
| JS1149-2 | D (37 mg/mL) | 93 | 201 | 380 | 2200 | 21 |

The starting material had essentially no species defined as fully sialylated (A2F, A2, A2F+bisecting GlcNAc). However, under several of the reaction conditions, greater than 90% full sialylation (sialylated on both arms of branched glycan) could be achieved. Reactions run on material directly dissolved in BIS-TRIS achieved significantly lower levels of fully sialylated IgG than when using buffer exchanged material even when the reactions using directly dissolved material were conducted at a higher protein concentration. This might be because the sodium chloride in the starting material has a negative impact on the sialylation reaction. Interestingly, the NaCl did not seem to significantly impact the sialylation of non-IgG proteins.

### Example 2: Cohn precipitate is about 65% IgG

Cohn fraction II/III sourced from Sigma-Aldrich was dialyzed into BIS-TRIS across a 10KDa MWCO filter and/or was additionally buffer exchanged into BIS-TRIS using a desalting column (G25).

LC-MS was used to quantify the relative abundance of proteins in the starting material, in Privigen®, a commercially available IVIG preparation, and in both the soluble fraction and the insoluble fractions of Cohn II/III after dialysis based on molecular weight corrected normalized peptide spectral matches (nPSMs).

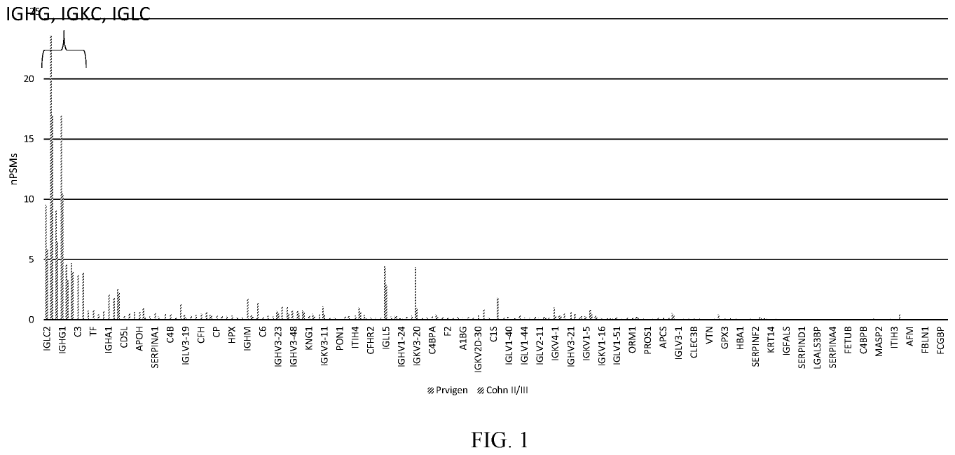

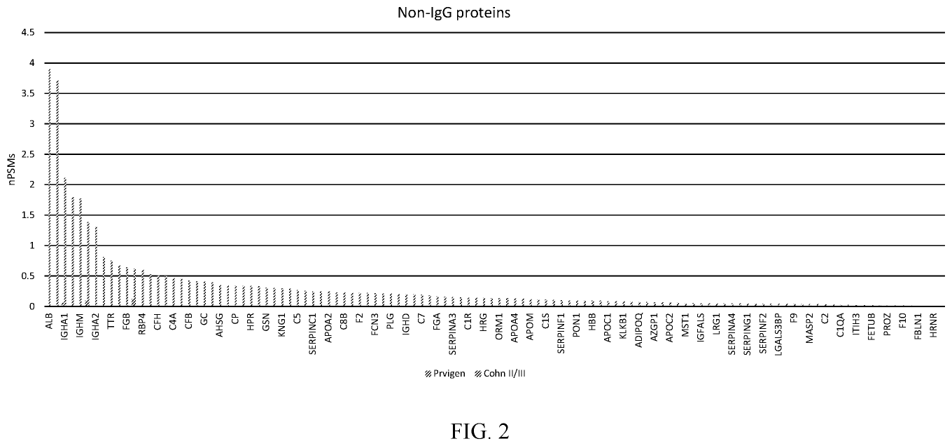

FIG. 1 shows the protein distribution in Cohn II/III and Privigen® and FIG. 2 shows the non-IgG protein distribution in Cohn II/III and Privigen®.

Based on normalized peptide spectral matches, Cohn II/III was about 65% IgG and 35% non-IgG, which is consistent with reported values. Privigen® was greater than 99% IgG. Thus, it can be appreciated that the material used in Example 1 to prepare hsIgG included a significant portion of protein that was other than IgG.

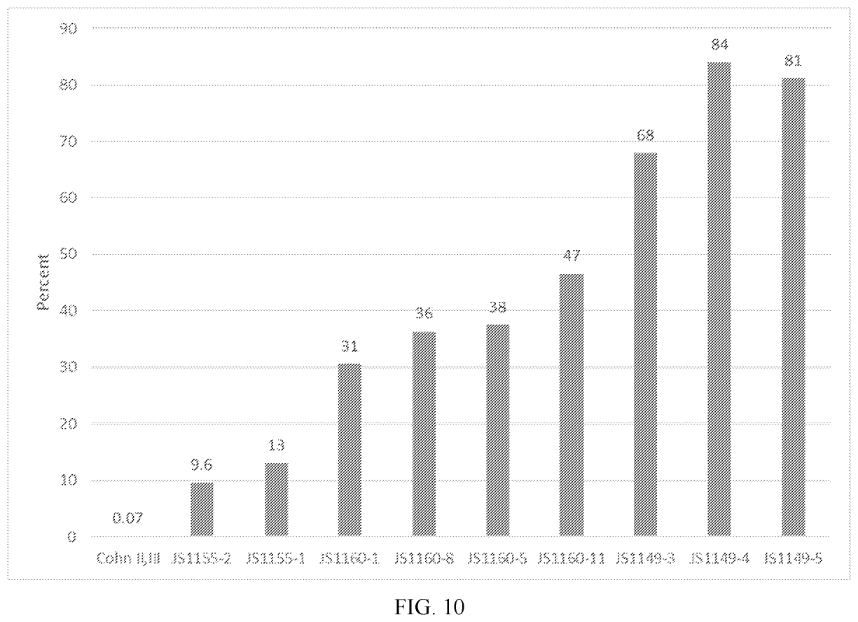

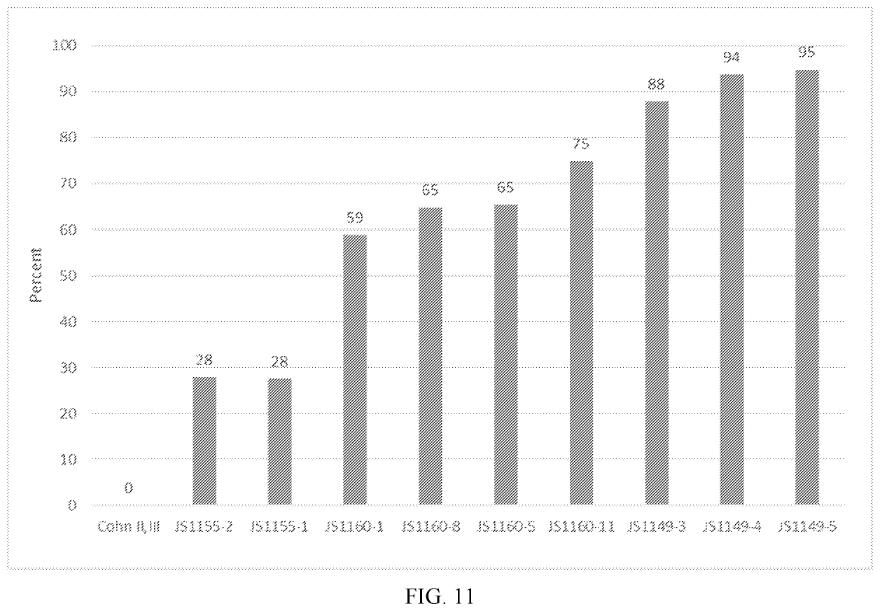

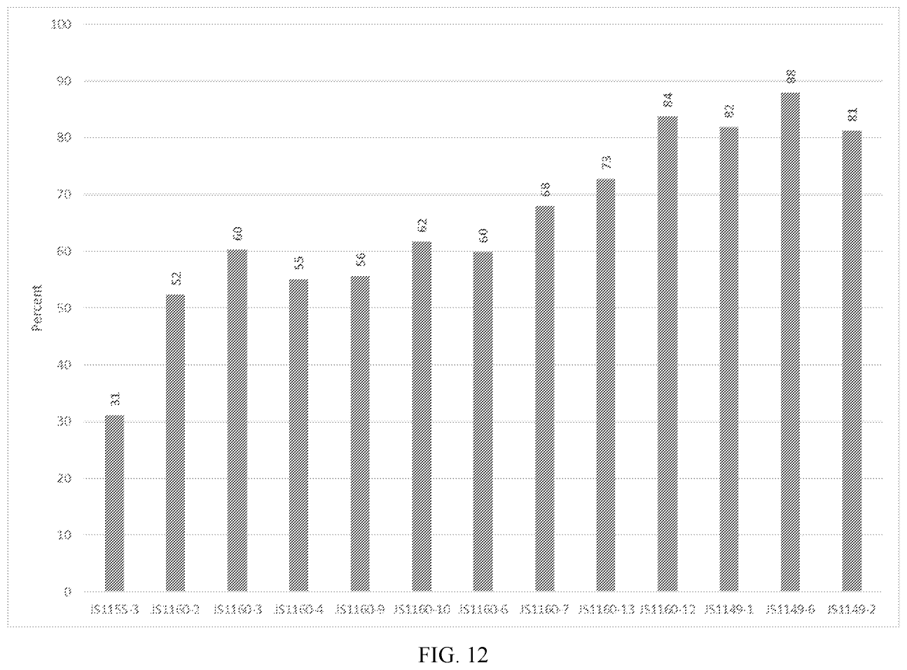

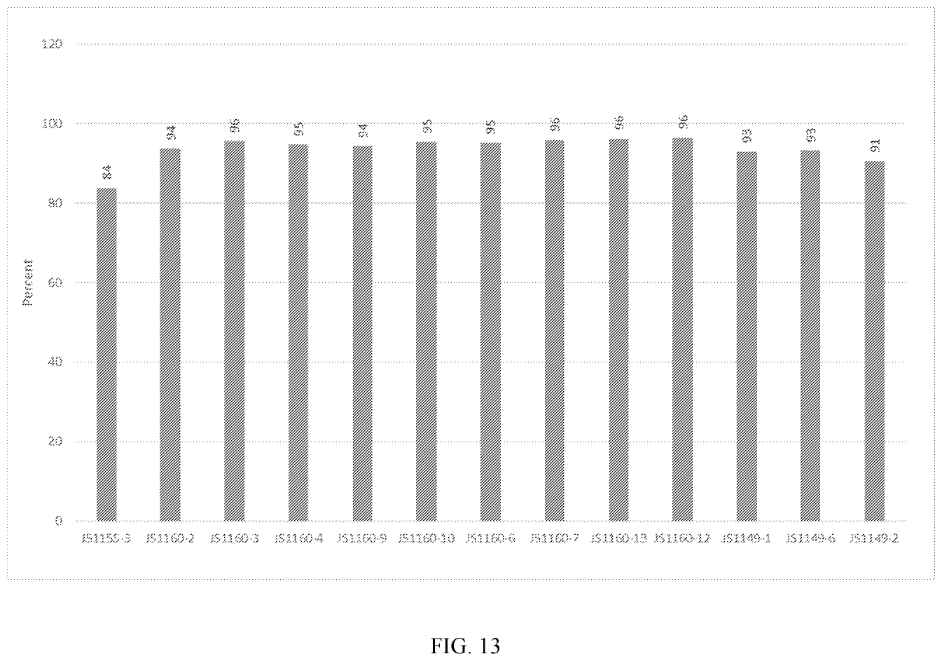

FIGS. 10-13 show the fractions of IgG2/3 (FIG. 10, FIG. 12) and IgG3/4 (FIG. 11, FIG. 13) branched glycans that are disialylated in the sialylation reactions shown in Tables 11 (FIG. 10, FIG. 11) and 12 (FIG. 12, FIG. 13) on Aldrich Cohn II, III material which was directly dissolved in BIS-TRIS. The glycans were quantified by LCMS analysis of glycopeptides from a trypsin digestion of the IgGs. Disialylated is defined as the sum A2F, A2F+bisect, A2.

### Example 3: Delipidation

Processing of Cohn II/III paste using no precipitation step but with delipidation prior to buffer exchange into BIS-TRIS was carried out as follows:

Cohn II/III paste was resuspended in water at 4° C. and the pH adjusted to 4.81 using 0.5 M acetic acid. Stirring was continued overnight at 4° C. The pH was adjusted to 7.00 using 2.4 M BIS-TRIS base buffer. After centrifugation the supernatant was filtered using 0.45 urn and then 0.22 urn filters and placed at 4° C. Lipid removal agent (LRA, Advanced Minerals Corp) and Celpure® P-100 (Advanced Minerals Corp) were added, the suspension stirred 1.5 h at ambient temperature, stirred 1.5 h at 4° C., and then 0.22 urn filtered. The conductivity was adjusted to 9.2 mS/cm using 5 M NaCl solution. Fumed silica 350-420 m²/g (Thermo Fisher) and Celpure® P-100 were added, the solution stirred 1 h at ambient temperature, 0.22 urn filtered, and then 0.1 urn filtered.

The solution was concentrated and buffer exchange by TFF (Pellicon 3 membrane, EMD Millipore) using six diavolumes 50 mM BIS-TRIS 7.20 buffer. After elution from the TFF system the material was further concentrated to 100 mg/mL (using an extinction coefficient of 1.37) in Vivaspin Turbo 15 devices (Sartorius). Designate material “F”.

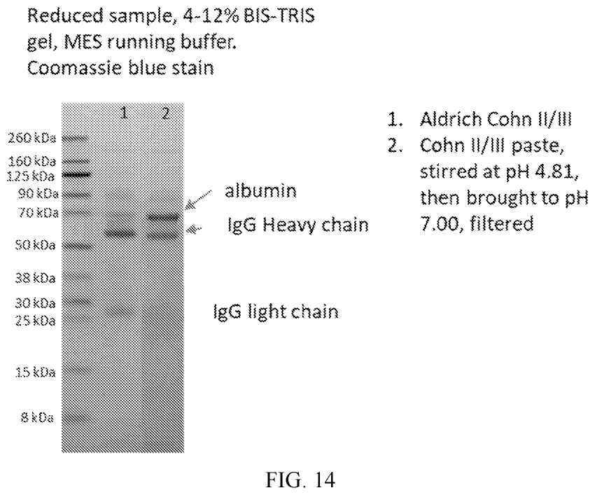

FIG. 14 shows an SDS-PAGE gel comparing the protein purity profile of Aldrich Cohn II/III material to a soluble fraction of Cohn II/III paste.

Cohn II/III material F obtained above was subjected to the galactosylation reaction.

| | | | | Fully | Fully | Fully |
|---|---|---|---|---|---|---|
| | mU | nmol | | galactosylated | galactosylated | galactosylated |
| | B4GalT/mg | UDP-Gal/mg | MnCl₂ | glycans IgG1 | glycans IgG2 | glycans |
| | IVIg | IVIg | (mM) | (%) | (%) | IgG3/4 (%) |
| JS1221-1 | 11 | 48 | 5.0 | 100 | 99 | 100 |

Reaction JS1212-1 material was then sialylated.

| | ST6 | nmol CMP- | Disialylated | Disialylated | Disialylated |
|---|---|---|---|---|---|
| | activity | NANA/mg | glycans | glycans | glycans |
| | (mU/mg) | IVIg total | IgG1 (%) | IgG2 (%) | IgG3/4 (%) |
| JS1221-3 | 18 | 150 | 82 | 63 | 100 |
| JS1221-4 | 36 | 300 | 94 | 83 | 100 |
| JS1221-5 | 54 | 450 | 97 | 90 | 100 |

Galactosylation (JS1212-1 stoichiometry) and sialylation (JS1212-4 stoichiometry) reactions were performed in which the two enzyme reactions were run concurrently or sequentially, as described below.

Sequential reaction:

Processed Cohn II/III (8.0 mL, 800 mg) in BIS-TRIS was mixed with 38 umol UDP-Gal, 9.0 U B4GalT, 5.0 mM manganese(II) chloride, and incubated at 37° C. 24 h. 120 umol CMP-NANA and 28.8 U ST6 were added and incubation continued. At 9 h an additional 120 umol CMP-NANA was added. Reaction was stopped at 31 h.

In the concurrent reaction all the reagents amount given above were mixed at T=0. At 9 h an additional 120 umol CMP-NANA was added. Reaction was stopped at 31 h total reaction time.

Both gave the same high levels of galactosylation and sialylation. Reaction on this material proceeded with less enzyme than Example 1. This is due in part to the high protein concentration achieved with low salt content.

| | | Disialylated | Disialylated | Disialylated |
|---|---|---|---|---|
| | Reaction | glycans | glycans | glycans |
| | Type | IgG1 (%) | IgG2 (%) | IgG3/4 (%) |
| JS1221-15 | Sequential | 95 | 85 | 100 |
| JS1221-16 | Concurrent | 94 | 82 | 100 |

SEQUENCES
(IgG1)
SEQ ID NO: 1
EEQYNSTYR
(IgG2/3)
SEQ ID NO: 2
EEQFNSTFR
(IgG3/4)
SEQ ID NO: 3
EEQYNSTFR
(IgG3/4)
SEQ ID NO: 4
EEQFNSTYR
(NP_001488.2 B4GALT1
[organism = Homo sapiens] [GeneID = 2683]
[isoform = 1])
SEQ ID NO: 5
TPLQGGSNSAAAIGQSSGELRTGGARPPPPLGASSQPRPGGDSSPVVDSGPGPASNL
TSVPVPHTTALSLPACPEESPLLVGPMLIEFNMPVDLELVAKONPNVKMGGRYAPRD
CVSPHKVAIIIPFRNRQEHLKYWLYYLHPVLORQQLDYGIYVINQAGDTIFNRAKLL
NVGFQEALKDYDYTCFVFSDVDLIPMNDHNAYRCFSQPRHISVAMDKFGFSLPYVQY
FGGVSALSKQQFLTINGFPNNYWGWGGEDDDIFNRLVFRGMSISRPNAVVGRCRMIR
HSRDKKNEPNPQRFDRIAHTKETMLSDGLNSLTYQVLDVQRYPLYTQITVDIGTPS
(NP_001365424.1 B4GALT1
[organism = Homo sapiens]
[GeneID = 2683] [isoform = 2])
SEQ ID NO: 6
GQSSGELRTGGARPPPPLGASSQPRPGGDSSPVVDSGPGPASNLTSVPVPHTTALSL
PACPEESPLLVGPMLIEFNMPVDLELVAKQNPNVKMGGRYAPRDCVSPHKVAIIIPF
RNRQEHLKYWLYYLHPVLQRQQLDYGIYVINQAGDTIFNRAKLLNVGFQEALKDYDY
TCFVFSDVDLIPMNDHNAYRCFSQPRHISVAMDKFGFSLPYVQYFGGVSALSKQQFL
TINGFPNNYWGWGGEDDDIFNRLVFRGMSISRPNAVVGRCRMIRHSRDKKNEPNPQR
FDRIAHTKETMLSDGLNSLTYQVLDVQRYPLYTQITVDIGTPS
(NP_001365425.1 B4GALT1
[organism = Homo sapiens]
[GeneID = 2683] [isoform = 3])
SEQ ID NO: 7
MRLREPLLSGSAAMPGASLORACRLLVAVCALHLGVTLVYYLAGRDLSRLPQLVGVS
TPLQGGSNSAAAIGQSSGELRTGGARPPPPLGASSQPRPGGDSSPVVDSGPGPASNL
TSVPVPHTTALSLPACPEESPLLVGPMLIEFNMPVDLELVAKQNPNVKMGGRYAPRD
CVSPHKVAIIIPFRNRQEHLKYWLYYLHPVLQRQQLDYGIYVINQAGDTIFNRAKLL
NVGFQEALKDYDYTCFVFSDVDLIPMNDHNAYRCFSQPRHISVAMDKFGFRLVFRGM
SISRPNAVVGRCRMIRHSRDKKNEPNPQRFDRIAHTKETMLSDGLNSLTYQVLDVQR
YPLYTQITVDIGTPS
(NP_001365426.1 B4GALT1
[GeneID = 2683] [isoform = 4])
[organism = Homo sapiens]
SEQ ID NO: 8
MRLREPLLSGSAAMPGASLORACRLLVAVCALHLGVTLVYYLAGRDLSRLPQLVGVS
TPLQGGSNSAAAIGQSSGELRTGGARPPPPLGASSQPRPGGDSSPVVDSGPGPASNL
TSVPVPHTTALSLPACPEESPLLVGPMLIEFNMPVDLELVAKQNPNVKMGGRYAPRD
CVSPHKVAIIIPFRNRQEHLKYWLYYLHPVLQRQQLDYGIYVINQYEKIRRLLW
SEQ ID NO: 9
MRLREPLLSGSAAMPGASLORACR
SEQ ID NO: 10
LLVAVCALHLGVTLVYYLAG
SEQ ID NO: 11
RDLSRLPQLVGVSTPLQGGSNSAAAIGQSSGELRTGGARPPPPLGASSQPRPGGDSS
PVVDSGPGPASNLTSVPVPHTTALSLPACPEESPLLVGPMLIEFNMPVDLELVAKQN
PNVKMGGRYAPRDCVSPHKVAIIIPFRNRQEHLKYWLYYLHPVLQRQQLDYGIYVIN
QAGDTIFNRAKLLNVGFQEALKDYDYTCFVFSDVDLIPMNDHNAYRCFSQPRHISVA
MDKFGFSLPYVQYFGGVSALSKQQFLTINGFPNNYWGWGGEDDDIFNRLVFRGMSIS
RPNAVVGRCRMIRHSRDKKNEPNPQRFDRIAHTKETMLSDGLNSLTYQVLDVQRYPL
YTQITVDIGTPS
(B4GalT)
SEQ ID NO: 12
GPASNLTSVPVPHTTALSLPACPEESPLLVGPMLIEFNMPVDLELVAKQNPNVKMGG
RYAPRDCVSPHKVAIIIPFRNRQEHLKYWLYYLHPVLQRQQLDYGIYVINQAGDTIF
NRAKLLNVGFQEALKDYDYTCFVFSDVDLIPMNDHNAYRCFSQPRHISVAMDKFGFS
LPYVQYFGGVSALSKQQFLTINGFPNNYWGWGGEDDDIFNRLVFRGMSISRPNAVVG
RCRMIRHSRDKKNEPNPQRFDRIAHTKETMLSDGLNSLTYQVLDVQRYPLYTQITVD
IGTPS
(B4GalT)
SEQ ID NO: 13
gssplldmGPASNLTSVPVPHTTALSLPACPEESPLLVGPMLIEFNMPVDLELVAKQ
NPNVKMGGRYAPRDCVSPHKVAIIIPFRNRQEHLKYWLYYLHPVLQRQQLDYGIYVI
NQAGDTIFNRAKLLNVGFQEALKDYDYTCFVFSDVDLIPMNDHNAYRCFSQPRHISV
AMDKFGFSLPYVQYFGGVSALSKQQFLTINGFPNNYWGWGGEDDDIFNRLVFRGMSI
SRPNAVVGRCRMIRHSRDKKNEPNPQRFDRIAHTKETMLSDGLNSLTYQVLDVQRYP
LYTQITVDIGTPSprdhhhhhhh
(NP_001340845.1
(NP_003023.1, NP_775323.1) ST6GAL1
[organism = Homo sapiens] [GeneID = 6480] [isoform = a])
SEQ ID NO: 14
MIHTNLKKKFSCCVLVFLLFAVICVWKEKKKGSYYDSFKLQTKEFQVLKSLGKLAMG
SDSQSVSSSSTQDPHRGRQTLGSLRGLAKAKPEASFQVWNKDSSSKNLIPRLQKIWK
NYLSMNKYKVSYKGPGPGIKFSAEALRCHLRDHVNVSMVEVTDFPFNTSEWEGYLPK
ESIRTKAGPWGRCAVVSSAGSLKSSQLGREIDDHDAVLRFNGAPTANFQQDVGTKTT
IRLMNSQLVTTEKRFLKDSLYNEGILIVWDPSVYHSDIPKWYQNPDYNFFNNYKTYR
KLHPNQPFYILKPQMPWELWDILQEISPEEIQPNPPSSGMLGIIIMMTLCDQVDIYE
FLPSKRKTDVCYYYQKFFDSACTMGAYHPLLYEKNLVKHLNQGTDEDIYLLGKATLP
GFRTIHC
(NP_775324.1 ST6GAL1
[organism = Homo sapiens] [GeneID = 6480]
[isoform = b])
SEQ ID NO: 15
MNSQLVTTEKRFLKDSLYNEGILIVWDPSVYHSDIPKWYQNPDYNFFNNYKTYRKLH
PNQPFYILKPOMPWELWDILQEISPEEIQPNPPSSGMLGIIIMMTLCDQVDIYEFLP
SKRKTDVCYYYQKFFDSACTMGAYHPLLYEKNLVKHLNQGTDEDIYLLGKATLPGFR
TIHC
SEQ ID NO: 16
MIHTNLKKK
SEQ ID NO: 17
FSCCVLVFLLFAVICVW
SEQ ID NO: 18
KEKKKGSYYDSFKLQTKEFQVLKSLGKLAMGSDSQSVSSSSTQDPHRGRQTLGSLRG
LAKAKPEASFQVWNKDSSSKNLIPRLQKIWKNYLSMNKYKVSYKGPGPGIKFSAEAL
RCHLRDHVNVSMVEVTDFPFNTSEWEGYLPKESIRTKAGPWGRCAVVSSAGSLKSSQ
LGREIDDHDAVLRFNGAPTANFQQDVGTKTTIRLMNSQLVTTEKRFLKDSLYNEGIL
IVWDPSVYHSDIPKWYQNPDYNFFNNYKTYRKLHPNQPFYILKPQMPWELWDILQEI
SPEEIQPNPPSSGMLGIIIMMTLCDQVDIYEFLPSKRKTDVCYYYQKFFDSACTMGA
YHPLLYEKNLVKHLNQGTDEDIYLLGKATLPGFRTIHC
(ST6Gal1)
SEQ ID NO: 19
AKPEASFQVWNKDSSSKNLIPRLQKIWKNYLSMNKYKVSYKGPGPGIKFSAEALRCH
LRDHVNVSMVEVTDFPFNTSEWEGYLPKESIRTKAGPWGRCAVVSSAGSLKSSQLGR
EIDDHDAVLRFNGAPTANFQQDVGTKTTIRLMNSQLVTTEKRFLKDSLYNEGILIVW
DPSVYHSDIPKWYQNPDYNFFNNYKTYRKLHPNQPFYILKPQMPWELWDILQEISPE
EIQPNPPSSGMLGIIIMMTLCDQVDIYEFLPSKRKTDVCYYYQKFFDSACTMGAYHP
LLYEKNLVKHLNQGTDEDIYLLGKATLPGFRTIHC
(ST6Gal1)
SEQ ID NO: 20
LOKIWKNYLSMNKYKVSYKGPGPGIKFSAEALRCHLRDHVNVSMVEVTDFPFNTSEW
EGYLPKESIRTKAGPWGRCAVVSSAGSLKSSQLGREIDDHDAVLRFNGAPTANFQQD
VGTKTTIRLMNSQLVTTEKRFLKDSLYNEGILIVWDPSVYHSDIPKWYQNPDYNFFN
NYKTYRKLHPNQPFYILKPOMPWELWDILQEISPEEIQPNPPSSGMLGIIIMMTLCD
QVDIYE
(ST6Gal1)
SEQ ID NO: 21
gssplldmlehhhhhhhhmAKPEASFQVWNKDSSSKNLIPRLQKIWKNYLSMNKYKV
SYKGPGPGIKFSAEALRCHLRDHVNVSMVEVTDFPFNTSEWEGYLPKESIRTKAGPW
GRCAVVSSAGSLKSSQLGREIDDHDAVLRFNGAPTANFQQDVGTKTTIRLMNSQLVT
TEKRFLKDSLYNEGILIVWDPSVYHSDIPKWYQNPDYNFFNNYKTYRKLHPNQPFYI
LKPQMPWELWDILQEISPEEIQPNPPSSGMLGIIIMMTLCDQVDIYEFLPSKRKTDV
CYYYQKFFDSACTMGAYHPLLYEKNLVKHLNQGTDEDIYLLGKATLPGFRTIHC

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of preparing a hypersialylated human immunoglobulin G (hsIgG) preparation, the method comprising:
(a) providing a composition comprising pooled human immunoglobulin G (IgG) wherein at least 5% or at least 10% wt/wt of protein in the composition is not IgG;
(b) adding β1,4-Galactosyltransferase I (β4GalT) and uridine 5′-diphosphogalactose (UDP-Gal), together or sequentially, to the composition to create a reaction mixture, wherein the reaction mixture is in a buffer;
(c) incubating the reaction mixture;
(d) adding ST6 beta-galactoside alpha-2,6-sialyltransferase 1 (ST6Gal1) and cytidine-5′-monophospho-N-acetylneuraminic acid (CMP-NANA), together or sequentially, to the reaction mixture; and
(e) incubating the reaction mixture, thereby creating the hsIgG preparation.

2. A method of preparing hypersialylated (hsIgG), the method comprising:
(a) providing a composition comprising pooled human IgG wherein at least 5% or at least 10% wt/wt of protein in the composition is not IgG in a buffer;
(b) incubating the composition in a reaction mixture comprising β1,4-Galactosyltransferase I (β4GalT), UDP-Gal, ST6Gal1 and CMP-NANA, in a buffer, thereby creating the hsIgG preparation.

3. A method of preparing hypersialylated (hsIgG), the method comprising:
(a) providing a composition comprising pooled human IgG wherein at least 5% or at least 10% wt/wt of protein in the composition is not IgG;
(b) combining the composition with β4GalT and ST6Gal to create a reaction mixture, wherein the reaction mixture is in a buffer and contains UDP-Gal and CMP-NANA;
and incubating the reaction mixture, thereby creating the hsIgG preparation.

4. The method of claim 1, wherein the buffer is selected from the group consisting of Bis(2-hydroxyethyl)amino0tris(hydroxymethyl)methane (BIS-TRIS), 3-(N-morpholino)propanesulfonic acid (MOPS), 2-(N-morpholino)ethanesulfonic acid (MES), 1,4-Piperazinediethanesulfonic acid (PIPES), N,N-Bis(2-hydroxyethyl)-2-aminoethanesulfonic acid (BES), 3-morpholino-2-hydroxypropanesulfonic acid (MOPSO), Triethanolamine (TEA), Piperazine-N—N′-bis(2-hydroxypropanesulfonic acid (POPSO), 4-(2-Hydroxyethyl)-1-piperazinepropanesulfonic acid (EPPS), and combinations thereof.

5. The method of claim 1, wherein providing a mixture of IgG antibodies includes (a) providing pooled plasma from at least 1000 human subjects; and (b) isolating a mixture of IgG antibodies from the pooled plasma.

6. (canceled)

7. The method of claim 1, wherein additional CMP-NANA is added to the reaction at a time after the first addition of CMP-NANA.

8-14. (canceled)

15. The method of claim 1, further comprising subjecting the hsIgG preparation to one or more purification steps.

16-36. (canceled)

37. The method of claim 1, wherein the reactions take place in BIS-TRIS at 10-500 mM pH 5.5-8.5.

38. The method of claim 1, wherein reaction mixtures comprise MnCl₂ at 1-20 mM.

39. The method of claim 1, wherein the initial concentration of UDP-Gal in the reaction mixture comprising UDP-Gal is 20-500 UDP-Gal/g IgG antibody.

40. The method of claim 1, wherein initial concentration of CMP-NANA in the reaction mixture comprising CMP-NANA is 100-3000 μmol CMP-NANA/g IgG antibody.

41-44. (canceled)

45. The method of claim 1, wherein the hsIgG preparation is further treated to removed ST6Gal1 and β4GalT.

46. The method of claim 1, wherein at least 50%, 55%, 60%, 65%, 70%, 75%, 80%, or 85% of the branched glycans in the hsIgG preparation have a sialic acid on both the α1,3 branch and the α1,6 branch.

47-58. (canceled)


SEQUENCE LISTING (21 sequences)

SEQ ID NO: 1 - protein, 9 aa, Artificial Sequence
  To quantify glycopeptides for IgG1
EEQYNSTYR

SEQ ID NO: 2 - protein, 9 aa, Artificial Sequence
  To quantify glycopeptides for IgG2/3
EEQFNSTFR

SEQ ID NO: 3 - protein, 9 aa, Artificial Sequence
  To quantify glycopeptides for IgG3/4
EEQYNSTFR

SEQ ID NO: 4 - protein, 9 aa, Artificial Sequence
  To quantify glycopeptides for IgG3/4
EEQFNSTYR

SEQ ID NO: 5 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 6 - protein, 385 aa, Homo sapiens
MPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 7 - protein, 357 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFRLVFRGMSISRPNAVVGRCRMIRHSRDKKNEPNPQRFDRIAHTKETMLSDGLNSLTYQVLDVQRYPLYTQITVDIGTPS

SEQ ID NO: 8 - protein, 225 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQYEKIRRLLW

SEQ ID NO: 9 - protein, 24 aa, Homo sapiens
MRLREPLLSGSAAMPGASLQRACR

SEQ ID NO: 10 - protein, 20 aa, Homo sapiens
LLVAVCALHLGVTLVYYLAG

SEQ ID NO: 11 - protein, 354 aa, Homo sapiens
RDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 12 - protein, 290 aa, Homo sapiens
GPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 13 - protein, 308 aa, Homo sapiens
GSSPLLDMGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPSPRDHHHHHHH

SEQ ID NO: 14 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 15 - protein, 175 aa, Homo sapiens
MNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 16 - protein, 9 aa, Homo sapiens
MIHTNLKKK

SEQ ID NO: 17 - protein, 17 aa, Homo sapiens
FSCCVLVFLLFAVICVW

SEQ ID NO: 18 - protein, 380 aa, Homo sapiens
KEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 19 - protein, 320 aa, Homo sapiens
AKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 20 - protein, 234 aa, Homo sapiens
LQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYE

SEQ ID NO: 21 - protein, 339 aa, Homo sapiens
GSSPLLDMLEHHHHHHHHMAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC